(19) United States
(12) Patent Application Publication
(10) Pub. No.: US 2021/0251864 A1
(43) Pub. Date: Aug. 19, 2021

(54) ECO EMULSIFIER

(71) Applicant: SYMRISE AG, Holzminden (DE)

(72) Inventors: Gerhard SCHMAUS, Höxter-Bosseborn (DE); Nikolas BUGDHAN, Holzminden (DE); Sabine LANGE, Holzminden (DE); Dietmar DROEGE, Ottenstein (DE); Vanessa SCHADE, Deensen (DE)

(21) Appl. No.: 17/273,464

(22) PCT Filed: Sep. 5, 2018
(86) PCT No.: PCT/EP2018/073899

§ 371 Date: Mar. 4, 2021

(51) Int. Cl. A61K 8/37; A61K 8/06; A61K 47/14; A61Q 19/10; A61Q 5/02

(52) U.S. Cl. CPC A61K 8/375; A61K 8/062; A61Q 5/02; A61Q 19/10; A61K 47/14

(57) ABSTRACT

Summary:

Described is an emulsifier comprising or consisting of a mixture of compound(s) of formula (I)

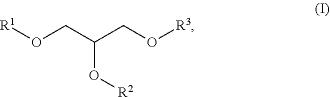

wherein in particular in each case:
R¹, R² and R³ each independently mean
(i) a hydrogen atom,
(ii) a fatty acid residue attached to the glycerol backbone by an ester bond, or
(iii) a citric acid residue attached to the glycerol backbone by an ester bond or an ether bond,
with the proviso that in each case at most one of the rests R¹, R² and R³ is a citric acid rest (iii) attached to the glycerol backbone by an ester bond or ether bond,
as well as corresponding salts and mixtures thereof.

Also described are an emulsifier precursor, processes for producing the emulsifier and the emulsifier precursor, the use of corresponding mixtures as emulsifiers, as well as emulsions based on the emulsifier precursors and emulsifiers, processes for producing the emulsions, the use of the emulsions in preparations and the corresponding preparations.

The present invention relates primarily to novel emulsifiers comprising or consisting of a mixture of compounds of formula (I)

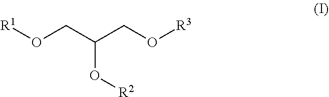

as described herein (for the meaning of R¹, R² and R³ see below), one or more different salt(s) of the compound(s) of formula (I) as described herein or one or more different compound(s) of formula (I) as described herein and one or more different salt(s) of the compound(s) of formula (I) as described herein. Hereinafter, the term “compound of formula (I)” as well as a salt of such compound or a plurality of such compounds or salts or mixtures thereof means a compound of formula (I) “as described herein” or a salt of such compound or a plurality of such compounds or salts or mixtures thereof.

In addition, the present invention relates to emulsifier precursors comprising or consisting of one or more compound(s) of formula (I) and/or one or more salt(s) of the compound(s) of formula (I).

Another aspect of the present invention relates to a process for producing the emulsifier according to the invention and the emulsifier precursor according to the invention.

Furthermore, the present invention relates to the use of mixtures of compound(s) of formula (I) according to the invention as emulsifier.

In addition, the present invention relates to emulsions based on one or more emulsifier precursor(s) according to the invention and/or one or more emulsifier(s) according to the invention, comprising or consisting of an aqueous phase and an oil phase, and a process for preparing the emulsions according to the invention.

Another aspect of the present invention relates to the use of one or more emulsions according to the invention in preparations for cleaning, cosmetic or pharmaceutical, preferably dermatological, preparations, preparations for consumption or nutrition or semi-finished goods, comprising one or more emulsifier precursor(s) according to the invention and/or one or more emulsifier(s) according to the invention and/or one or more emulsion(s) according to the invention as well as these preparations according to the invention.

Further aspects of the present invention as well as particularly advantageous embodiments will be apparent from the following description, the examples and the patent claims.

Emulsifiers based on natural fats/oils and citric acid have been known for a long time. They are used in the food industry as emulsifiers, complexing agents (inter alia to support the effect of antioxidants) and carriers with the designation E472c (citric acid esters of mono- and diglycerides of edible fatty acids or citrem) and can be found inter alia in cakes, biscuits, puff pastries, bread, sausage products, ice cream and desserts, confectionery and baking fats (Schuster, G., et al., Emulgatoren fur Lebensmittel, Berlin, Heidelberg, New York, Tokyo: Springer 1985, S. 107-114).

Emulsifiers are auxiliary agents for the production and stabilisation of emulsions, which in a narrower sense can be described as surface-active substances or surfactants and are usually present as oily to waxy, but also powdery substances. Emulsifiers reduce the interfacial tension between the phases of emulsions and stabilise the formed emulsion. The structural characteristic of emulsifiers is their amphiphilic molecular structure. A molecule of such a compound has at least one group with affinity for substances of strong polarity and at least one group with affinity for non-polar substances.

Emulsions are disperse systems of two or more immiscible liquids or immiscible liquid phases. One of the liquid phases forms the dispersion medium (also: outer, continuous or coherent phase), in which the other phase (also: inner or disperse phase) is distributed in the form of fine droplets. The particle diameter of the particles, which are preferably assumed to be spherical in an idealised view or whose size is given as the size of an equivalent sphere of the same diameter (“equivalent sphere”), varies in the process. Most emulsions show non-uniform particle size and are polydisperse. Most natural and technical emulsions consist of water and oil or fat as immiscible phases. An O/W emulsion or oil-in-water emulsion is a fat-water mixture whose continuous phase is aqueous. Accordingly, an O/W emulsifier is an emulsifier that stabilises an O/W emulsion or contributes to its stability.

EP 1 641 904 B1 discloses an O/W emulsifier containing 70 to 90 wt.-% of glyceryl oleate citrate and 10 to 30 wt.-% of a viscosity modifier, which allows stable, cold-adjustable and low-viscosity, in particular sprayable, O/W emulsions to be produced.

JP 2012 031250 A and US 2011/0273646 A1 disclose glycerylol citrate fatty acid esters of caprylic acid, stearic acid and oleic acid as an ingredient for the preparation of protective films for optical polarizers, in particular optical polarizers in liquid crystal-based liquid crystal display (LCD) devices with the aim of providing LCD devices particularly well resistant to temperature and humidity.

Despite a large number of known emulsifiers, there is still a need, among others in the food processing industry, for new emulsifiers which, in addition to their primary properties as solubilisers for immiscible phases, preferably have additional positive properties such as negligible or no toxicity and/or (increased) skin compatibility or mucous membrane compatibility and/or provide improved properties in terms of increased emulsifiability (with lower consumption) and/or a broader area of application.

The primary object of the present invention was to provide new emulsifiers, preferably emulsifiers which are distinguished from the prior art by additional improved properties, e.g. in the sense of increased emulsifiability (with lower consumption) and/or a broader field of application and/or the suitability for enrichment of the emulsifier, and particularly preferably by negligible or no toxicity, and/or (increased) skin compatibility or (increased) mucous membrane compatibility.

Further objects underlying the present invention result from the following descriptions and the patent claims.

The aforementioned primary object of the present invention is solved according to the invention by an emulsifier comprising or consisting of a mixture of compounds of formula (I)

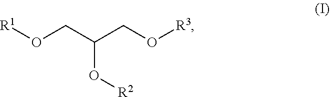

wherein in each case applies:
R¹, R² and R³ each independently mean
(i) a hydrogen atom,
(ii) a fatty acid residue attached to the glycerol backbone by an ester bond, or
(iii) a citric acid residue attached to the glycerol backbone by an ester bond or an ether bond

with the proviso that in each case at most one of the rests R¹, R² and R³ is a citric acid rest (iii) attached to the glycerol backbone by an ester bond or ether bond, one or more different salt(s) of the compound(s) of formula (I) or one or more different compound(s) of formula (I) and one or more different salt(s) of the compound(s) of formula (I),

wherein the proportion of the total amount of compounds of formula (I) and salts of the compound of formula (I) having one or two fatty acid residues (ii) attached to the glycerol backbone by an ester bond is higher than 40 wt.-%, higher than 50 wt.-%, higher than 60 wt.-%, higher than 70 wt.-%, higher than 80 wt.-%, higher than 90 wt.-% or higher than 95 wt.-%, based on the total weight of the emulsifier,

wherein the proportion of the total amount of compounds of formula (I) and salts of the compounds of formula (I) having exactly one citric acid residue (iii) attached to the glycerol backbone by an ester bond or ether bond and at least one fatty acid residue (ii) attached to the glycerol backbone by an ester bond is higher than 20 wt.-%, higher than 21 wt.-%, higher than 22 wt.-%, higher than 23 wt.-%, higher than 24 wt.-% or higher than 25 wt.-%, based on the total weight of the emulsifier and,

wherein the proportion of the total amount of compounds of formula (I) and/or one or more salt(s) of the compound(s) of formula (I) having one or more fatty acid residue(s) (ii) attached to the glycerol backbone by one or more ester bond(s), wherein the, one, more or all of the fatty acid residue(s) (ii) is/are independently selected from the group consisting of the fatty acid residue of caproic acid, the fatty acid residue of caprylic acid, the fatty acid residue of capric acid, the fatty acid residue of lauric acid and the fatty acid residue of myristic acid, is higher than 55 wt.-%, preferably higher than 70 wt.-% or higher than 75 wt.-%, particularly preferably higher than 90 wt.-%, relative to the total weight of the compounds of formula (I),

and/or

the proportion of the total amount of compounds of formula (I) and/or one or more salt(s) of the compound(s) of formula (I) having one or more fatty acid residue(s) (ii) attached to the glycerol backbone by one or more ester bond(s), wherein the, one, more or all fatty acid residue(s) (ii) is/are independently selected from the group consisting of the fatty acid residue of lauric acid and the fatty acid residue of myristic acid, is higher than 50 wt.-%, preferably higher than 55 wt.-%, particularly preferably higher than 60 wt.-%, based on the total weight of the compounds of formula (I),

and/or

wherein for one or more compounds of formula (I) applies that the fatty acid residue(s) (ii) is/are independently selected from the group consisting of the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, is lower than 40 wt.-%, preferably lower than 35 wt.-%, particularly preferably lower than 30 wt.-%, based on the total weight of the compounds of formula (I).

In a preferred embodiment of the emulsifier, the mixture of compounds of formula (I) comprises 5-20% compounds of formula (I) having exactly one fatty acid residue and at most one citric acid residue and 20-50% compounds of formula (I) having exactly two fatty acid residues and at most one citric acid residue and 25-60% compounds of formula (I) having exactly three fatty acid residues.

The compounds of formula (I) to be used according to the invention have an amphiphilic molecular structure.

A “fatty acid residue” in the sense of the present invention (see for example the meaning of residues R¹, R² and R³ as described herein; alternative (ii)) is to be understood as a structural component for which the following formula applies:

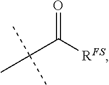

wherein the dotted line marks the bond which independently links one of the rests R¹, R² or R³ in consideration of the previous descriptions and stipulations regarding the compounds of formula (I) to one of the oxygen atoms marked by “◯” in the compounds of formula (I), and wherein RFS is according to the nomenclature known to the skilled person a univalent radical selected from the group consisting of alkyl radicals, alkadienyl radicals and alkatrienyl radicals.

“Citric acid” or the “citric acid residue” derived therefrom within the meaning of the invention comprises citric acid (2-hydroxypropane-1,2,3-tricarboxylic acid, in particular CAS: 77-92-9 or InChlKey: KRKNYBCHXYNGOX-UHFFFAOYSA-N) or the residue derived therefrom as well as the diastereomers thereof or the residues derived therefrom and the enantiomers thereof or the residues derived therefrom, in particular isocitric acid (3-carboxy-2-hydroxy-pentane-1,5-diacid, in particular InChlKey: ODBLHEXUDAPZAU-FONMRSAGSA-N) or the rest derived therefrom and the enantiomers thereof or rests derived therefrom.

A citric acid residue bound by an ester bond within the meaning of the present invention (see for example the meaning of residues R¹, R² and R³ as described herein; alternative (iii)) is to be understood, shown here for example for citric acid, as a structural component for which one of the following formulae (iii-a) or (iii-b) applies:

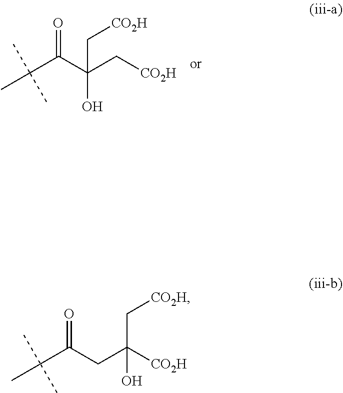

wherein the dotted line marks the bond which independently links one of the rests R¹, R² or R³ in consideration of the previous descriptions and stipulations regarding the compounds of formula (I) to one of the oxygen atoms marked by “◯” in each of the compounds of formula (I).

A citric acid residue bound by an ether bond within the meaning of the present invention (see for example the meaning of residues R¹, R² and R³ as described herein; alternative (iii)) is to be understood, shown here for example for citric acid, as a structural component for which the following formula (iii-c) applies:

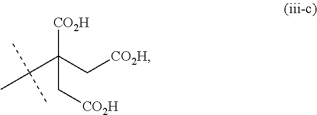

wherein the dotted line marks the bond which independently links one of the rests R¹, R² or R³ in consideration of the previous descriptions and stipulations regarding the compounds of formula (I) to one of the oxygen atoms marked by “◯” in each of the compounds of formula (I).

A “salt” of the compounds of formula (I) in the sense of the present invention is preferably an alkali or alkaline earth salt of (partially) deprotonated compounds of formula (I) at the OH or COOH groups, if present in the compounds of formula (I).

An emulsifier according to the invention as described herein is not known from the prior art. For example, patent EP 1 641 904 B1 does not address the possibility of using fatty acid residues other than those described therein, in particular shorter-chain fatty acid residues, as components of glycerylol citrate fatty acid esters. Instead, only the positive effect of caprylic or capric triglycerides as viscosity modifiers on both the stability of the O/W emulsifier and the emulsions prepared therewith is described. Documents JP 2012 031250 A and US 2011/0273646 A1, for example, also provide no reference to the use of shorter chain fatty acid residues, other than those mentioned therein, in the disclosed glycerylol citrate fatty acid esters.

For the purposes of the present invention, “shorter-chain fatty acid residues” are preferably residues of the fatty acids as defined below. Preferably, shorter-chain fatty acid residues are residues selected from the group consisting of the corresponding residues of fatty acids having 6 to 14 carbon atoms, in particular caproic acid, caprylic acid, capric acid, lauric acid and myristic acid. Particularly preferred are shorter-chain fatty acid residues selected from the group consisting of the corresponding residues of fatty acids having 12 to 14 carbon atoms, in particular lauric acid and myristic acid. “Longer chain fatty acid residues” in the sense of the present invention are preferably residues of the fatty acids selected from the group consisting of the corresponding residues of the fatty acids having more than 14 carbon atoms, in particular palmitic acid, palmitoleic acid, stearic acid, oleic acid and linolenic acid.

The “proportion of the total amount of compounds of the formula (I) and salts of the compounds of the formula (I) relative to the total weight of the emulsifier” is preferably determined by comparison of mass spectra with reference spectra, taking into account the elution position and the UV spectra. High-resolution time-of-flight (TOF) mass spectra can be used to determine the molar mass of the compound and, if necessary, a molecular formula. The sample is separated by liquid chromatography and detected by mass spectrometry (MS). The generated mass spectrum enables identification of the individual components. For further identification, it is possible to generate an MS spectrum of the substance and to carry out detection via light or laser light scattering. Alternatively, a gravimetric analysis is also applicable. Minor deviations in the determination of the weight percentages, e.g. due to different measuring methods, are acceptable within the scope of the present invention and are not relevant for the practicability of the objects of the invention described herein.

Similarly, “dimers” of compounds of formula (I) and/or of salts of the compounds of formula (I) may be present in the emulsifier according to the invention, wherein these compounds comprise a fatty acid residue, a citric acid residue and a dimeric glycerol backbone linked via an ether bond, or two fatty acid residues, a citric acid residue and a dimeric glycerol backbone linked via an ether bond, as well as the diastereomers of these compounds and their enantiomers. In addition, preliminary experiments have shown indications that these dimers possibly contribute synergistically to the emulsifying effect according to the invention of the compounds of formula (I) according to the invention.

The same as in the previous paragraph applies to compounds with exactly two citric acid residues (iii) R¹, R² and R³ each independently attached to the glycerol backbone by ester and/or ether bonds.

In the sense of the present invention, according to one embodiment, emulsifiers in which the proportion of the total amount of compounds of the formula (I) and salts of the compounds of the formula (I) having exactly one citric acid residue (iii) attached to the glycerol backbone by an ester bond or ether bond is higher than 85 wt.-%, particularly preferably in which said proportion is higher than 95 wt.-%, based on the total weight of the emulsifier, are preferred.

In another embodiment of the present invention, the emulsifier is one comprising or consisting of one or more different compound(s) of formula (I)

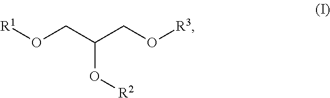

wherein in each case applies:
R¹, R² and R³ each independently mean
(i) a hydrogen atom,
(ii) a fatty acid residue attached to the glycerol backbone by an ester bond, or
(iii) a citric acid residue attached to the glycerol backbone by an ester bond or an ether bond,

with the proviso that in each case at most one of the rests R¹, R² and R³ is a citric acid rest (iii) attached to the glycerol backbone by an ester bond or ether bond, one or more different salt(s) of the compound(s) of the formula (I) or one or more different compound(s) of the formula (I) and one or more different salt(s) of the compound(s) of the formula (I),

wherein the proportion of the total amount of compounds of formula (I) and salts of the compounds of formula (I) having exactly one citric acid residue (iii) attached to the glycerol backbone by an ester bond or ether bond is higher than 20 wt.-%, higher than 21 wt.-%, higher than 22 wt.-%, higher than 23 wt.-%, higher than 24 wt.-% or higher than 25 wt.-%, based on the total weight of the emulsifier.

In a further preferred embodiment of the emulsifier according to the invention, the mixture of compounds of the formula (I) consists of 5-20% compounds of the formula (I) with exactly one fatty acid residue and/or exactly one citric acid residue and of 20-50% compounds of the formula (I) with exactly two fatty acid residues and/or exactly two fatty acid residues and exactly one citric acid residue and 25-50% compounds of the formula (I) with exactly three fatty acid residues and the remainder for 100% of compounds of the formula (I) with exactly three hydrogen atoms and/or citric acid.

In one embodiment of the emulsifier according to the invention, the emulsifier is a mixture consisting of or comprising one or more compound(s) of formula (I), wherein the fatty acid residue(s) (ii) is/are in consideration of the previous descriptions and stipulations regarding the compounds of formula (I) independently selected from the group consisting of the fatty acid residue of caproic acid of formula (ii-a):

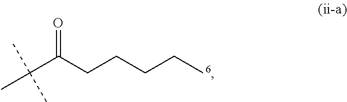

the fatty acid residue of caprylic acid having the formula (ii-b):

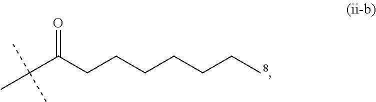

the fatty acid residue of capric acid having the formula (ii-c):

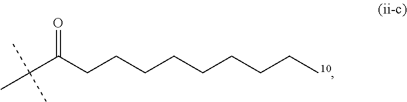

the fatty acid residue of lauric acid with the formula (ii-d):

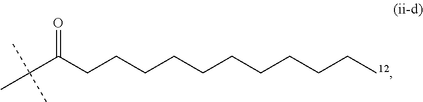

the fatty acid residue of myristic acid with the formula (ii-e):

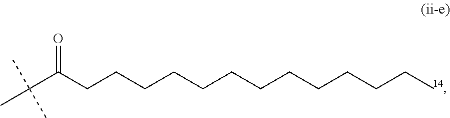

the fatty acid residue of palmitic acid with the formula (ii-f):

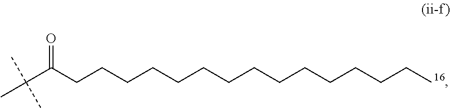

the fatty acid residue of palmitoleic acid with the formula (ii-g):

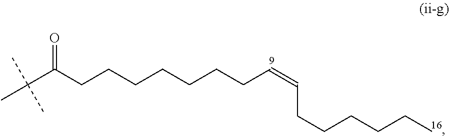

the fatty acid residue of stearic acid having the formula (ii-h)

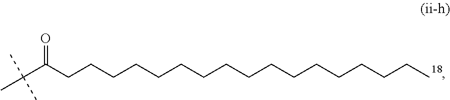

the fatty acid residue of oleic acid with the formula (ii-i):

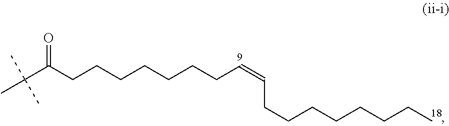

the fatty acid residue of linoleic acid with the formula (ii-j):

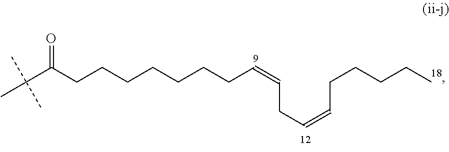

and the fatty acid residue of linolenic acid with the formula (ii-k):

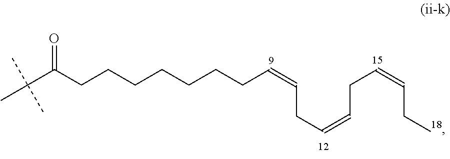

preferably from the group consisting of the fatty acid residue of lauric acid and the fatty acid residue of myristic acid, wherein the dotted line of the fatty acid residues (ii-a) to (ii-k) marks the bond linking each of the residues R¹, R² or R³ independently of one another, in consideration of the previous descriptions and stipulations regarding the compounds of formula (I), to one of the oxygen atoms marked by “◯” in the compounds of formula (I).

In case of any discrepancies between a structural formula shown and the name given to the compound or the respective structural element, or if the name does not fully comply with the convention, the respective shown structural formula or the respective shown structural element shall apply.

The fatty acid residues (ii-a) to (ii-k) described above as constituents, i.e. one or more of the residues R¹, R²and R³ of the emulsifiers according to the invention have advantageous solubilising properties over or a stronger emulsifying effect than previously known emulsifiers from the prior art and are furthermore suitable for use in a broader field of application than previously known emulsifiers, wherein the above-mentioned object is advantageously solved.

Particularly preferred is an emulsifier according to the invention, wherein the proportion of the total amount of compounds of formula (I) and/or one or more salt(s) of the compound(s) of formula (I) with one or more fatty acid residue(s) (ii) attached to the glycerol backbone by one or more ester bond(s), wherein the, one, more or all fatty acid residue(s) (ii) is/are independently selected from the group consisting of the fatty acid residue of caproic acid, the fatty acid residue of caprylic acid, the fatty acid residue of capric acid, the fatty acid residue of lauric acid and the fatty acid residue of myristic acid, is higher than 55 wt.-%, preferably higher than 70 wt.-% or higher than 75 wt.-%, particularly preferably higher than 90 wt.-%, based on the total weight of the compounds of formula (I), and/or

the proportion of the total amount of compounds of formula (I) and/or one or more salt(s) of the compound(s) of formula (I) having one or more fatty acid residue(s) (ii) attached to the glycerol backbone by one or more ester bond(s), in which the fatty acid residue(s) (ii) is/are independently selected from the group consisting of the fatty acid residue of lauric acid and the fatty acid residue of myristic acid, is higher than 50 wt.-%, preferably higher than 55 wt.-%, more preferably higher than 60 wt.-%, based on the total weight of the compounds of formula (I).

Surprisingly, it was found that emulsifiers according to the invention with a high proportion of short-chain fatty acid residues bound by ester bonds and mentioned previously, in particular fatty acid residues of caproic acid, fatty acid residues of caprylic acid, fatty acid residues of capric acid, fatty acid residues of lauric acid and fatty acid residues of myristic acid, exhibit improved emulsifying properties. This provides reduced particle sizes and a broader field of application of the emulsifiers according to the invention.

Particularly preferred is an emulsifier according to the invention or a mixture of emulsifiers according to the invention described herein, wherein for one or more compounds of formula (I) applies that the fatty acid residue(s) (ii) is/are independently selected from the group consisting of the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, is lower than 40 wt.-%, preferably lower than 35 wt.-%, particularly preferably lower than 35 wt.-%, relative to the total weight of the compounds of formula (I).

It was surprisingly found that emulsifiers according to the invention with a low proportion of longer-chain fatty acid residues bound by ester bonds and mentioned previously, in particular the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, provide improved emulsifying properties. This provides reduced particle sizes and a broader field of application of the emulsifiers according to the invention compared to known emulsifiers.

The following describes processes according to the invention for the production of the emulsifiers according to the invention described herein.

Accordingly, a further aspect of the present invention relates to a process for producing an emulsifier according to the invention, comprising or consisting of the following steps:

(A) reacting a fatty acid composition consisting of or comprising one or more fatty acids and/or one or more fatty acid residues, preferably wherein the, one, more or all fatty acid(s) is/are selected from the group consisting of caproic acid, caprylic acid, capric acid, lauric acid, myristic acid, palmitic acid, palmitoleic acid, stearic acid, oleic acid, linoleic acid and linolenic acid and/or one or more fatty acid residue(s), preferably wherein the, one, more or all fatty acid residue(s) (ii) is/are selected from the group consisting of from the group consisting of the fatty acid residue of caproic acid, the fatty acid residue of caprylic acid, the fatty acid residue of capric acid, the fatty acid residue of lauric acid, the fatty acid residue of myristic acid, the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, particularly preferably consisting of or comprising at least lauric acid and/or myristic acid and/or the fatty acid residue of lauric acid and/or the fatty acid residue of myristic acid, and/or preferably wherein the fatty acid composition is selected from the group consisting of an oil, a fat, a fatty oil or mixtures thereof or comprising such, with glycerol and one or more catalytic bases,

(B) adding citric acid, one or more salt(s) of citric acid or a mixture of citric acid and one or more salt(s) of citric acid, to the mixture obtained in step (A), and

(C) optionally: precipitating the mixture obtained in step (B) with one or more solvents to enrich the compound(s) of formula (I), and

(D) optionally: purifying the mixture obtained in step (B) or (C) by using one or more solvents selected from the group consisting of halogenated, polar, protic polar, protic non-polar, “green” solvents and mixtures thereof or comprising such, and/or performing one or more purification procedures selected from the group consisting of filtration, ultra- or nanofiltration, adsorption and absorption techniques, extraction, recrystallisation and chromatographic methods and combinations thereof,

wherein in the fatty acid composition used in step (A), the proportion of the total amount of fatty acids selected from the group consisting of caproic acid, caprylic acid, capric acid, lauric acid and myristic acid and/or fatty acid residues selected from the group consisting of the fatty acid residue of caproic acid, the fatty acid residue of caprylic acid, the fatty acid residue of capric acid, the fatty acid residue of lauric acid and the fatty acid residue of myristic acid is higher than 45 wt.-%, preferably higher than 55 wt.-%, based on the total weight of the fatty acid composition,

preferably wherein in the fatty acid composition used in step (A) the proportion of the total amount of
lauric acid and/or the fatty acid residue of lauric acid is higher than 20 wt.-%, preferably higher than 35 wt.-%, more preferably higher than 40 wt.-%, preferably based on the total weight of the fatty acid composition,

and/or
myristic acid and/or the fatty acid residue of myristic acid is higher than 5 wt.-%, preferably higher than 10 wt.-%, more preferably higher than 15 wt.-%, based on the total weight of the fatty acid composition.

The process described above is suitable for the production of emulsifiers according to the invention, wherein, depending on whether and to what degree the purification steps mentioned above are carried out, an emulsifier according to the invention or mixtures of emulsifiers according to the invention are obtained, which will be discussed further below, in particular in the examples. Accordingly, the process described above is suitable for providing the emulsifiers according to the invention, which are suitable for solving the technical object.

“Reacting” in the sense of the present invention means bringing the respective reagents into contact in a container or a plurality of such containers suitable for chemical reaction, preferably under controlled conditions comprising pressure and temperature, with the aim of obtaining an intermediate product or product, which can thus be used either directly or after purification or is used in further reactions.

A “fatty acid composition” in the sense of the present invention is a compound or a mixture of compounds suitable for providing either the aforementioned fatty acid residues and/or the corresponding fatty acids. According to the invention, a fatty acid composition thus comprises one or more oil(s), one or more fat(s), one or more fatty oil(s), one or more fatty acid(s) for the structure of which (ii-x) applies:

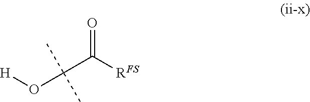

or mixtures thereof, wherein the part to the right of the dotted line of (ii-x) preferably means one of the structural elements of the parts to the right of the dotted line of compounds (ii-a) to (ii-k) according to the invention. With regard to R^{FS}, the above also applies.

“Precipitation” in the sense of the present invention means a method of precipitating a dissolved substance wholly or partially as an insoluble precipitate in the form of crystals, flakes or droplets by the addition of suitable substances (precipitating agents). It does not matter whether the precipitant changes the substance's chemical composition or not. The resulting precipitates of precipitated solids are initially mostly microcrystalline or amorphous. With further contact with the supernatant solution (mother liquor), an enlargement of the particles and possibly a transformation into more stable crystal modifications often takes place in the course of time due to recrystallisation.

“Purification” in the sense of the present invention means all processes, individually or in their entirety, which serve to obtain the products of the invention of a certain degree of purity by separating them from the impurities, i.e. products other than those of the invention or other desired compounds. Purification processes include, for example, dissolution and precipitation, the use of adsorption processes and chromatography processes, the use of electrophoresis, melting, freezing, normal solidification, crystallisation, sublimation, growth processes and other transport reactions, (fractional) distillation, rectification and many other separation processes.

Accordingly, another embodiment of the present invention relates to a process for the production of an emulsifier according to the invention, comprising or consisting of the following steps:

(A) reacting a fatty acid composition consisting of or comprising one or more fatty acids and/or one or more fatty acid residues, preferably wherein the, one, more or all fatty acid(s) is/are selected from the group consisting of caproic acid, caprylic acid, capric acid, lauric acid, myristic acid, palmitic acid, palmitoleic acid, stearic acid, oleic acid, linoleic acid and linolenic acid and/or one or more fatty acid residue(s), preferably wherein the, one, more or all fatty acid residue(s) (ii) is/are selected from the group consisting of the fatty acid residue of caproic acid, the fatty acid residue of caprylic acid, the fatty acid residue of capric acid, the fatty acid residue of lauric acid, the fatty acid residue of myristic acid, the fatty acid residue of palmitic acid, the fatty acid residue of palmitoleic acid, the fatty acid residue of stearic acid, the fatty acid residue of oleic acid, the fatty acid residue of linoleic acid and the fatty acid residue of linolenic acid, particularly preferably consisting of or comprising at least lauric acid and/or myristic acid and/or the fatty acid residue of lauric acid and/or the fatty acid residue of myristic acid, and/or preferably wherein the fatty acid composition is selected from the group consisting of an oil, a fat, a fatty oil or mixtures thereof or comprising such, with glycerol and one or more catalytic bases,

(B) adding citric acid, one or more salt(s) of citric acid or a mixture of citric acid and one or more salt(s) of citric acid, to the mixture obtained in step (A), and

(C) optionally: precipitating the mixture obtained in step (B) with one or more solvents to enrich the compound(s) of formula (I), and

(D) optionally: purifying the mixture obtained in step (B) or (C) by using one or more solvents selected from the group consisting of halogenated, polar, protic polar, protic non-polar, “green” solvents and mixtures thereof or comprising such, and/or performing one or more purification procedures selected from the group consisting of filtration, ultra- or nanofiltration, adsorption and absorption techniques, extraction, recrystallisation and chromatographic methods and combinations thereof.

Preferred is a process according to the invention, wherein in the fatty acid composition used in step (A) the proportion of the total amount of fatty acids selected from the group consisting of caproic acid, caprylic acid, capric acid, lauric acid and myristic acid and/or fatty acid residues selected from the group consisting of the fatty acid residue of caproic acid, the fatty acid residue of caprylic acid, the fatty acid residue of capric acid, the fatty acid residue of lauric acid and the fatty acid residue of myristic acid, is higher than 45 wt.-%., preferably higher than 55 wt.-%, based on the total weight of the fatty acid composition, preferably wherein in the fatty acid composition used in step (A) the proportion of the total amount of lauric acid and/or the fatty acid residue of lauric acid is higher than 20 wt.-%, preferably higher than 35 wt.-%, particularly preferably higher than 40 wt.-%, based on the total weight of the fatty acid composition and/or myristic acid and/or the fatty acid residue of myristic acid is higher than 5 wt.-%, preferably higher than 10 wt.-%, particularly preferably higher than 15 wt.-%, based on the total weight of the fatty acid composition.

Surprisingly, it was found that the process described above leads to advantageous emulsifiers according to the invention with a high proportion of short-chain fatty acid residues bound by ester bonds and mentioned herein, in particular fatty acid residues of caproic acid, fatty acid residues of caprylic acid, fatty acid residues of capric acid, fatty acid residues of lauric acid and fatty acid residues of myristic acid, improved emulsifying properties (see above).

For the purpose of determining the proportion of the total amount of fatty acids in the fatty acid composition, both bound fatty acid residues and freely present fatty acid are preferably to be assigned to the total amount of the respective fatty acid. The determination of the respective proportions is preferably performed chromatographically or gravimetrically as described above. For example, the chromatographic method described herein may be carried out using the following equipment, (consumable) materials and parameters: LC instrument: Waters Acquity UPLC, MS instrument: Bruker micrOTOF Q-II, column: Kinetex RP-C18, 1.7 μm (100×2.1 mm), mobile phase A: water+0.1 wt.-% formic acid, mobile phase B: acetonitrile+0.09 wt.-% formic acid, flow: 0.55 mL/min, temperature: 50° C.

The upper limit of the proportion of the total amount of fatty acids from the group consisting of caproic acid, caprylic acid, capric acid, lauric acid and myristic acid and/or fatty acid residues from the group consisting of the fatty acid residue of caproic acid, the fatty acid residue of caprylic acid, the fatty acid residue of capric acid, the fatty acid residue of lauric acid and the fatty acid residue of myristic acid used in step (A) is preferably at most 90 wt.-%, particularly preferably at most 85 wt.-%, based on the total weight of the fatty acid composition. These values result from the composition, preferably the natural composition, of the fatty acid composition used according to the invention.

Particularly preferred in the sense of the present invention is one of the processes according to the invention described above, wherein in step (A) glycerol is used for the reaction in an amount of 0.5 to 5 mol equivalents, preferably in an amount of 1.0 to 2.0 mol equivalents, particularly preferably in an amount of 1.25 to 1.75 mol equivalents, based on the total amount of substance of fatty acid composition.

Surprisingly, it has been shown that the use of the aforementioned reagents in the aforementioned concentration range has an advantageous effect on the production of emulsifiers according to the invention.

Advantageous in the sense of the present invention is one of the processes according to the invention described above, wherein the catalytic base(s) from step (A) is/are used in an amount of 0.01 to 0.5 mol equivalents, preferably in an amount of 0.025 to 0.1 mol equivalents, particularly preferably in an amount of 0.05 to 0.1 mol equivalents, based on the total amount of substance of the fatty acid composition and/or wherein the catalytic base(s) of step (A) is/are independently selected from the group consisting of amine bases, amino acids, Brønsted bases, Lewis bases, alkali salts, alkaline earth salts and mixtures thereof, preferably from the group consisting of sodium hydroxide, potassium hydroxide and mixtures thereof.

Surprisingly, it has been found that the use of the said reagents or in the concentration range mentioned herein has an advantageous effect on the production of emulsifiers according to the invention. The use of one or more of the said reagents in the concentration range mentioned herein has been found to be particularly preferred.

Particularly preferred in the sense of the present invention is one of the processes according to the invention described herein, wherein step (A) is carried out at a temperature in the range from 150 to 300° C., preferably in the range from 175 to 275° C., particularly preferably in the range from 200 to 250° C., and optionally under vacuum conditions.

A “temperature in range” from a lower limit to an upper limit in the sense of the present invention means that the temperature is within the specified range during the entire reaction period or during the substantial part of the reaction period. This includes both isothermal reaction conditions and reaction conditions with temperature profiles where the temperature is substantially within the specified limits. A brief departure from the temperature range for a non-substantial period of time is not opposed to this.

The same applies analogously to a “pressure in the range” from a lower limit to an upper limit.

“Vacuum” as used in the present invention means the presence of a pressure in a range of 900 mbar or less, preferably in a range of 100 to 700 mbar, more preferably in a range of 200 to 500 mbar.

Unless explicitly stated otherwise, the process or the individual, several or all process steps are carried out independently of each other at standard conditions, i.e. standard temperature and/or standard pressure. If values are given for only one of the parameters, standard conditions apply in this case for the other parameter.

Surprisingly, it has been found that carrying out step (A) at a temperature in the aforementioned range has an advantageous effect on the production of emulsifiers according to the invention. Furthermore, emulsifier precursors according to the invention are characterised by an improved colour and odour profile. This improved colour and odour profile is particularly advantageous for the final colour and odour impression of the emulsifiers according to the invention based thereon or of the further products according to the invention. It is assumed by the inventors that this feature is related to a suitable ratio according to the invention of mono- and diglycerides or of mono- to diglycerides in the underlying fatty acid composition, which remains essentially unchanged by the process steps mentioned.

Particularly preferred in the sense of the present invention is one of the processes according to the invention described herein, wherein in step (B) a total amount of citric acid and/or one or more salt(s) thereof is added in the range of from 0.1 to 2.0 parts by weight, preferably in the range of from 0.25 to 1.5 parts by weight, more preferably in the range of from 0.25 to 1.0 parts by weight, based on the total weight of the mixture obtained in step (A).

The use of citric acid in the concentration range mentioned above has surprisingly proven to be advantageous for the production of emulsifiers according to the invention.

Particularly preferred in the sense of the present invention is one of the processes according to the invention described above, wherein a temperature in the range from 50 to 200° C., preferably in the range from 100 to 175° C., particularly preferably in the range from 120 to 160° C., and optionally vacuum conditions are set for the reaction taking place in step (B) after the addition of citric acid and/or one or more salt(s) thereof.

With regard to the vacuum conditions of step (B), the same applies as for the vacuum conditions of step (A). In particular, it has been shown that the combination of vacuum conditions in both step (A) and step (B) has an advantageous effect on the production of emulsifiers according to the invention.

Surprisingly, it has been found that carrying out step (B) at a temperature in the aforementioned range has an advantageous effect on the production of emulsifiers according to the invention. As described herein, emulsifiers according to the invention are advantageously characterised by an improved colour and odour profile.

Advantageously, for the purposes of the present invention, is one of the methods of the invention described herein, wherein the, one, more or all solvent(s) in step (C), if present, is/are preferably selected from the group consisting of pentane, hexane, heptane, cyclopentane, cyclohexane, tetrachloromethane, diisopropyl ether, toluene, benzene, diethyl ether, dichloromethane, chloroform, acetone, dioxane, tetrahydrofuran, 2-methylthetrahydrofuran, tert-butyl methyl ether, ethyl acetate, dimethyl sulphoxide, acetonitrile, benzonitrile, pyridine, 2-propanol, ethanol, methanol, acetic acid, formic acid, water and mixtures thereof.

A “solvent” in the sense of the present invention describes a substance which can bring other substances to solution by physical means, in the narrower sense an inorganic or organic liquid which is capable of dissolving other gaseous, liquid or solid substances.

Preferred solvents or mixtures of solvents may include: Water, lower alcohols (ethanol, methanol), acetone, 1,4-dioxane, tetrahydrofuran, tert-butyl methyl ether, propylene glycol, glycerol, aliphatic esters of aliphatic alcohols (such as ethyl acetate, triacetin), ethers (e.g. diethyl ether), alkanes (e.g. hexane, heptane), chlorine-containing solvents (e.g. chloroform, dichloromethane) and aromatic solvents (e.g. benzene, toluene), vegetable oils or fats, or supercritical carbon dioxide or mixtures of the solvents mentioned herein.

A “green solvent” in the sense of the present invention fulfils, in addition to the conditions mentioned above, the criterion of minimising the environmental impact resulting from the use of the green solvent. In addition to health and safety issues, the assessment of the environmental impact also includes energy considerations, for example with regard to the energy required to reach the boiling point (C. Capello et al., What is a green solvent? A comprehensive framework for the environmental assessment of solvents, Green Chemistry, 2007, 9, 927-934).

Particularly preferred in the sense of the present invention is one of the methods described above, wherein the fatty acid composition is selected from the group consisting of babassu oil, palm kernel oil, coconut oil, macaúba oil, neutral oil, microalgae oil and mixtures thereof.

“Babassu oil” within the meaning of the present invention is a vegetable fat, preferably obtained from the seeds of the babassu or cusipalm (Attalea speciosa). According to the invention, it can be used in semi-solid, tallowy form, as a liquid, clear to yellowish oil or in refined form. More preferred is the use as a light yellowish oil. Particularly preferred is the use in the form of a refined colourless, odourless oil with a melting point close to room temperature.

Surprisingly, it has been found that the use of said oils or mixtures thereof as fatty acid compositions according to the invention, preferably based on natural raw materials, has an advantageous effect on the preparation of emulsifiers according to the invention.

Other oils that can be used in the sense of the present invention are cohune (palm) oil (Orbignya cobune/Attalea cohune), murumuru butter (Astrocaryum murumuru), Laurel oil (laurus nobilis), Nutmeg oil (Myristica fragrans), African nutmeg oil (Pycnanthus angolensis), Peach kernel oil (Pejibaye palm, Bactris gasipaes), Indaiá oil (Attalea dubia).

The process according to the invention described herein or its embodiments described as preferred or preferred are used according to the invention for the production of emulsifiers, emulsifier precursors and mixtures thereof according to the invention.

Accordingly, another aspect of the present invention relates to an emulsifier precursor obtained by or obtainable by purifying the mixture obtained from step (B) according to one of the methods of the invention described above using one or more solvents selected from the group consisting of halogenated, polar, protic polar, protic non-polar, “green” solvents and mixtures thereof, and/or by performing one or more purification processes selected from the group consisting of filtration, ultra- or nanofiltration, adsorption and absorption techniques, extraction, recrystallisation and chromatographic methods and combinations thereof.

The emulsifier precursor may comprise residues of the reactants and of the catalytic base(s) used, in an amount of >5% citric acid, >5% glycerol and/or >10% triglycerides.

Preferred is a further enrichment of the compounds of formula (I) contained in the emulsifier according to the invention obtained or obtainable from steps (B), (C) or (D) to concentrations in the range from 25 to 100 wt.-%, preferably in the range from 45 to 100 wt.-%, more preferably in the range from 75 to 100 wt.-% or in the range from 80 to 100 wt.-% or in the range from 85 to 100 wt.-% or in the range from 90 to 100 wt.-%, more preferably in the range from 95 to 100 wt.-% or in the range from 97 to 100 wt.-% or in the range from 99 to 100 wt.-% by one or more enrichment steps, based on the total weight of the emulsifier according to the invention. For the purposes of the present invention, “enrichment” is to be understood as using one or more purification processes described herein.

Preferred in the sense of the present invention is one of the emulsifiers according to the invention described above, obtained by or obtainable by one of the processes according to the invention described above.

Particularly preferably in the sense of the present invention, one of the emulsifiers according to the invention described above is obtained by or obtainable by one of the processes described above, wherein the process comprises step (C), and optionally wherein the proportion of the total amount of compounds of formula (I) and one or more salt(s) of the compound(s) of formula (I) having exactly one citric acid residue (iii) attached to the glycerol backbone by an ester bond or ether bond is higher than 50 wt.-%, preferably higher than 85 wt.-%, based on the total weight of the emulsifier, and preferably wherein the ratio of the proportions of the total amount of compounds of formula (I) and one or more salt(s) of compound(s) of formula (I) having exactly one fatty acid residue attached to the glycerol backbone by an ester bond to the total amount of compounds of formula (I) and one or more salt(s) compound(s) of formula (I) having exactly two fatty acid residues attached to the glycerol backbone by ester bonds is in the range of 1:1.5 to 1:3, preferably in the range from 1:1.75 to 1:2, particularly preferably in the range from 1:2 to 1:2.5, based on the weight of these compounds.

It has been surprisingly shown that emulsifiers in which the proportion of the total amount of compounds of the formula (I) and one or more salt(s) of the compound(s) of the formula (I) having exactly one citric acid residue (iii) attached to the glycerol backbone by an ester bond or ether bond is higher than 50 wt.-%, preferably higher than 85 wt.-%, relative to the total weight of the emulsifier, have particularly advantageous properties.

The particular suitability for the preparation of emulsions of reduced particle size is, according to own investigations, related to the ratio of the proportions of the total amount of compounds of formula (I) and one or more salt(s) of compound(s) of formula (I) with exactly one fatty acid residue attached by an ester-bond to the glycerol backbone to the total amount of compounds of formula (I) and one or more salt(s) of compound(s) of formula (I) having exactly two fatty acid residues attached to the glycerol backbone by ester bonds in the range from 1:1.5 to 1:3, preferably in the range from 1:1.75 to 1:2, particularly preferably in the range from 1:2 to 1:2.5, based on the weight of these compounds.

Another aspect of the present invention relates to the use of a mixture of compounds of formula (I) as defined above as an emulsifier.

A further embodiment of the present invention relates to the use of a mixture comprising or consisting of one or more of the aforementioned different compound(s) of formula (I) according to the invention, one or more different salt(s) thereof or one or more different compound(s) of formula (I) and one or more different salt(s) thereof, wherein the proportion of the total amount of compounds of formula (I) and salts of the compounds of formula (I) having exactly one citric acid residue (iii) attached to the glycerol backbone by an ester bond or ether bond is higher than 20 wt.-%, higher than 21 wt.-%, higher than 22 wt.-%, higher than 23 wt.-%, higher than 24 wt.-% or higher than 25 wt.-%, based on the total weight of the mixture, as emulsifier.

Details of emulsions according to the invention, methods according to the invention for the production of such emulsions and preparations according to the invention comprising the emulsifier precursors, emulsifiers or mixtures thereof described above are described below.

The above explanations apply to the compounds of the formula (I) and/or salts of the compounds of the formula (I) or mixtures thereof contained in the emulsions and preparations according to the invention.

Accordingly, the present invention further relates to an emulsion comprising or consisting of an oil phase containing one or more of the emulsifier precursor(s) according to the invention and as described herein and/or one or more of the emulsifier(s) according to the invention and as described herein, or a mixture thereof, and an aqueous phase.

The constituents of the oil phase of the emulsions according to the invention can advantageously be selected from the group of esters of saturated and/or unsaturated, branched and/or unbranched alkane carboxylic acids having a chain length of 3 to 30 C atoms and/or their salts and saturated and/or unsaturated, branched and/or unbranched alcohols having a chain length of 3 to 30 C atoms and/or their salts, from the group of esters of aromatic carboxylic acids and saturated and/or unsaturated, branched and/or unbranched alcohols having a chain length of 3 to 30 carbon atoms and/or their salts. Preferably, the oil phase comprises components selected from the group consisting of cetyl stearyl alcohol, pentaerythrityl distearate, cetearyl octanoate, Persea Gratissima (avocado) oil, caprylic or caprin triglceride and dimethicone. Particularly preferably, the oil phase comprises ingredients selected from the group consisting of cetylstearyl alcohol in a proportion of 0.0001 wt.-% to 50 wt.-%, preferably in a proportion of 0.01 wt.-% to 5 wt.-%, more preferably in a proportion of 0.5 wt.-% to 1.5 wt.-%, pentaerythrityl distearate in a proportion of 0.0001 wt.-% to 50 wt.-%, preferably in a proportion of 0.01 wt.-% to 5 wt.-%, particularly preferably in a proportion of 0.7 wt.-% to 1.7 wt.-%, cetearyl octanoate in a proportion of 0.0001 wt.-% to 50 wt.-%, preferably in a proportion of 1 wt.-% to 10 wt.-%, particularly preferably in a proportion of 3.5 wt.-% to 4.5 wt.-%, Persea Gratissima (avocado) oil in a proportion of 0.0001 wt.-% to 50 wt.-%, preferably in a proportion of 0.01 wt.-% to 6 wt.-%, particularly preferably in a proportion of 1.5 wt.-% to 2.5 wt.-%, caprylic or caprin triglceride in a proportion of 0.0001 wt.-% to 50 wt.-%, preferably in a proportion of 0.01 wt.-% to 15 wt.-%, particularly preferably in a proportion of 5 wt.-% to 7 wt.-%, and dimethicone in a proportion of 0.0001 wt.-% to 50 wt.-%, preferably in a proportion of 0.001 wt.-% to 3 wt.-%, particularly preferably in a proportion of 0.05 wt.-% to 1 wt.-%, based on the total weight of the emulsion according to the invention.

Furthermore, constituents of the oil phase can advantageously be selected from the group of branched and unbranched hydrocarbons and hydrocarbon waxes, silicone oils, dialkyl ethers, the group of saturated or unsaturated, branched or unbranched alcohols, as well as fatty acid triglycerides, namely triglycerol esters of saturated and/or unsaturated, branched and/or unbranched alkane carboxylic acids with a chain length of 8 to 24, in particular 12 to 18, carbon atoms. The fatty acid triglycerides can be advantageously selected, for example, from the group of synthetic, semi-synthetic and natural oils, e.g. olive oil, sunflower oil, soya oil, peanut oil, rapeseed oil, almond oil, palm oil, coconut oil, palm kernel oil and the like.

The aqueous phase of emulsions within the meaning of this invention optionally advantageously contains water-soluble plant extracts, alcohols, diols or polyols (lower alkyl), as well as their ethers, preferably ethanol, isopropanol, propylene glycol, glycerol, ethylene glycol monoethyl or monobutyl ether, propylene glycol monomethyl, monoethyl or monobutyl ether, diethylene glycol monomethyl or monoethyl ether and analogous products, furthermore alcohols (lower alkyl), e.g. ethanol, 1,2,2-diol or polyols (lower alkyl) e.g. ethanol, 1,2-propanediol, glycerol. Preferably, the aqueous phase comprises ingredients selected from the group consisting of water, demineralised water, hydroxyacetophenone and glycerol. Particularly preferably, the aqueous phase comprises constituents selected from the group consisting of hydroxyacetophenone in an amount of from 0.0001 wt.-% to 50 wt.-%, preferably in an amount of from 0.01 wt.-% to 5 wt.-%, more preferably in an amount of from 0.2 wt.-% to 1.2 wt.-%, and hydroxyacetophenone in an amount of from 0.0001 wt.-% to 50 wt.-%, preferably in a proportion of from 0.01 wt.-% to 10 wt.-%, particularly preferably in a proportion of from 2.5 wt.-% to 3.5 wt.-%, based on the total weight of the emulsion according to the invention.

It has been shown that the emulsions according to the invention are characterised by improved emulsifying properties compared to previously known emulsions and thus advantageously solve the underlying problem.

Preferred in the sense of the present invention is an emulsion according to the invention, further comprising one or more different compound(s) for reducing or increasing the viscosity of the emulsion.

Viscosity is the property, especially of a liquid, to resist the mutual laminar displacement of two adjacent layers. Accordingly, viscosity can also be understood as resilience or internal friction. For the specific adjustment of viscosity and the achievement of a defined consistency of emulsions or preparations, these can contain, for example, thickening agents on an organic (alginate, tragacanth, xanthan, modified celluloses, carrageenans, etc.) and/or inorganic (bentonite, pyrogenic silicic acid, magnesium aluminium silicates, etc.) basis.

Preferably, an emulsion according to the invention comprises one or more thickening agents which may advantageously be selected from the group consisting of silicon dioxide, aluminium silicates, polysaccharides or derivatives thereof, e.g. hyaluronic acid, xanthan gum, hydroxyropylmethylcellulose, carbomer (Ultrez-10), each individually or in combination. Preferably, the emulsion according to the invention comprises ingredients selected from the group consisting of xanthan gum, carbomer (Ultrez-10) and mixtures thereof. Particularly preferably, an emulsion according to the invention comprises ingredients selected from the group consisting of xanthan gum in a proportion of from 0.0001 wt.-% to 5 wt.-%, preferably in a proportion of from 0.01 wt.-% to 3 wt.-%, more preferably in a proportion of from 0.05 wt.-% to 0.6 wt.-%, and carbomer (Ultrez-10) in a proportion of 0.0001 wt.-% to 5 wt.-%, preferably in a proportion of 0.01 wt.-% to 3 wt.-%, particularly preferably in a proportion of 0.05 wt.-% to 0.7 wt.-%, based on the total weight of the emulsion according to the invention.

The present invention also relates to a use of an emulsion according to the invention in a preparation for cleaning, a cosmetic or pharmaceutical, preferably a dermatological, preparation, a preparation for consumption or nutrition, or in a semi-finished product for the preparation of such a preparation.

A cosmetic or pharmaceutical, preferably dermatological preparation, within the meaning of the present invention is preferably a preparation which, inter alia, preferably serves cosmetic skin care. Cosmetic skin care is primarily understood to mean strengthening or restoring the natural function of the skin as a barrier against environmental influences (e.g. dirt, chemicals, microorganisms) and against the loss of endogenous substances (e.g. water, natural fats, electrolytes), as well as supporting its horny layer in its natural regenerative capacity when damage occurs. If the barrier properties of the skin are disturbed, this can lead to increased resorption of toxic or allergenic substances or to infestation by microorganisms and, as a consequence, to toxic or allergic skin reactions. The aim of skin care is also to compensate for the loss of oil and water from the skin caused by daily washing. This is especially important when the natural regeneration capacity is not sufficient. In addition, skin care products should protect against environmental influences, especially sun and wind, and delay skin ageing.

Preferably, pharmaceutical preparations within the meaning of the invention are understood to be preparations which, for example, are in present the form of capsules, tablets (uncoated as well as coated tablets, e.g. enteric coatings), lozenges, granules, pellets, solid mixtures, dispersions in liquid phases, as emulsions, as powders, as solutions, as pastes or as other preparations which can be swallowed or chewed, and which are used as medicaments available on prescription, in pharmacies or otherwise, or as food supplements.

Medicinal topical compositions, as further examples of pharmaceutical preparations, usually contain one or more medical drug(s) in effective concentration(s). For the sake of simplicity, reference is made to the legal provisions of the Federal Republic of Germany (e.g. Cosmetics Regulation, Foodstuffs and Medicinal Products Act) for a clear distinction between cosmetic and medicinal use and corresponding products.

The cosmetic and pharmaceutical preparations within the meaning of the present invention may contain excipients such as those commonly used in such preparations, for example. preservatives, antioxidants, vitamins, bactericides, perfumes, substances to prevent foaming, dyes, pigments having a colouring effect, thickeners, surfactants, emollients, emulsifiers, moisturising and/or humectant substances, moisturisers, fats, oils, waxes, plant extracts or other common ingredients such as alcohols, lower alkyl alcohols, polyols, lower alkyl polyols, polymers, foam stabilisers, complexing agents, electrolytes, organic solvents, propellants, silicones or silicone derivatives.

The emulsifiers according to the invention can advantageously be incorporated into cosmetic and/or pharmaceutical, in particular dermatological, preparations which are composed in the usual way and serve for cosmetic and/or dermatological light protection, furthermore for the treatment, care and cleansing of the skin and/or hair and as a make-up product in decorative cosmetics. Accordingly, depending on their structure, such preparations may be used, for example, as a skin protection cream, cleansing milk, sun protection lotion, nourishing cream, day or night cream, etc. For example, such preparations may be in the form of emulsion, lotion, milk, cream, hydrodispersion gel, balm, spray, foam, hair shampoo, hair care product, hair conditioner, roll-on, stick or make-up.

Preparations for consumption or nutrition in the sense of the present invention are e.g. baked goods (e.g. bread, dry cookies, cake, further pastries), sweets (e.g. chocolates, chocolate bar products, further bar products, fruit gums, hard and soft caramels, chewing gum), alcoholoic or non-alcoholic drinks (e.g. coffee, tea, wine, wine containing drinks beer, beer containing drinks, liquers, hard liquers, brandy, fruit containing lemonades, isotonic drinks, refreshing dreinks, nectars, fruit and vegetable juices, fruit or vegetable juice preparations), instand drinks (e.g. instant cocoa drinks, instant tea drinks, instant coffee drinks, instant fruit drinks), meat products (e.g. ham, fresh or raw sausage preparations, seasoned or marinated fresh or cured meat products), eggs or egg products (dried egg, egg white, egg yolk), cereal products (e.g. breakfast cereals, cereal bars, pre-cooked ready-to-eat rice products), dairy products (e.g. Milk drinks, buttermilk drinks, milk ice cream, yoghurt, kefir, cream cheese, soft cheese, hard cheese, dried milk powder, whey, butter, buttermilk, partially or wholly hydrolysed milk protein-containing products), products made from soya protein or other soya bean fractions (e.g. (e.g. soy milk and products made from it, fruit drinks with soy protein, preparations containing soy lecithin, fermented products such as tofu or tempeh or products made from them), fruit preparations (e.g. jams, fruit ice cream, fruit sauces, fruit fillings), vegetable preparations (e.g. ketchup, sauces).(e.g. ketchup, sauces, dried vegetables, frozen vegetables, pre-cooked vegetables, boiled vegetables), snacks (e.g. baked or deep-fried crisps or potato dough products, extrudates based on maize or peanuts), fat- and oil-based products or emulsions thereof (e.g. mayonnaise, remoulade).(e.g. mayonnaise, tartar sauce, dressings), other ready-made meals and soups (e.g. dry soups, instant soups, pre-cooked soups), spices, seasoning mixtures and in particular seasonings, which are used for example in the snack sector. The preparations within the meaning of the invention may also serve as semi-finished goods for the production of further preparations for nutrition or enjoyment. The preparations within the meaning of the invention may also be in the form of capsules, tablets (uncoated as well as coated tablets, e.g. enteric coatings), lozenges, granules, pellets, mixtures of solids, dispersions in liquid phases, as emulsions, as powders, as solutions, as pastes or as other preparations which can be swallowed or chewed as food supplements.

Preferably, the preparations according to the invention may contain an aroma composition to round off and refine the taste and/or smell of the preparation. Suitable aroma compositions contain, for example, synthetic, natural or nature-identical flavour, aroma and taste substances. Preferred aroma compositions include, for example, vanillin, ethyl vanillin, ethyl vanillin isobutyrate (3-ethoxy-4-isobutyryloxybenzaldehyde), furaneol (2,5-dimethyl-4-hydroxy-3(2H)-furanone), and derivatives (e.g. homofuraneol, 2-ethyl-4-hydroxy-5-methyl-3(2H)-furanone), homofuronol (2-ethyl-5-methyl-4-hydroxy-3(2H)-furanone and 5-ethyl-2-methyl-4-hydroxy-3(2H)-furanone), maltol and derivatives (e.g. ethylmaltol), coumarin and derivatives, gamma-lactones (e.g. gamma-undecalactone, gamma-nonalactone), delta-lactones (e.g. 4-methyldeltalactone, massoilactone, deltadecalactone, tuberolactone), methyl sorbate, diacetyl, 4-hydroxy-2(or 5)-ethyl-5(or 2)-methyl-3(2H)furanone, 2-hydroxy-3-methyl-2-cyclopentenone, 3-hydroxy-4,5-dimethyl-2(5H)-furanone, fruit esters and fruit lactones (e.g. Acetic acid n-butyl ester, acetic acid isoamyl ester, propionic acid ethyl ester, butyric acid ethyl ester, butyric acid n-butyl ester, butyric acid isoamyl ester, 3-methylbutyric acid ethyl ester, n-hexanoic acid ethyl ester, n-hexanoic acid allyl ester, n-hexanoic acid n-butyl ester, n-octanoic acid ethyl ester, ethyl 3-methyl-3-phenyl glycidate, ethyl 2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenylacetaldehyde.

In the light of the above, it is clear that the present invention also relates to a cleaning preparation, cosmetic or pharmaceutical, preferably dermatological, preparation, a preparation for consumption or nutrition, preferably a preparation selected from the groups described above, or semi-finished product comprising one or more emulsifier precursor(s) according to the invention or one or more emulsifier(s) according to the invention or one or more emulsion(s) according to the invention.

A cleaning preparation in the sense of the present invention is preferably a preparation for cleaning surfaces (including glass, ceramics, granite, metal (in particular stainless steel), untreated wood, treated wood) in the household and/or industrial sector.

The preparations for consumption or nutrition according to the invention are regularly products which are intended to be introduced into the human oral cavity, to remain there for a certain time and then either to be consumed (for example, ready-to-eat food) or to be removed from the oral cavity. These products include any product or substance intended to be ingested by humans in a processed, partially processed or unprocessed state. Orally consumable preparations may also be products added to food during its manufacture, processing or treatment and intended to be placed in the human oral cavity. Accordingly, such preparations (here then as semi-finished products) may in turn be contained in (further) preparations for consumption or nutrition, which are ready for use or consumption, (preparations for consumption or nutrition, which are ready for use or consumption, are, in the context of the present text, in particular foodstuffs, especially ready-to-eat foodstuffs).

“Semi-finished product” in the sense of the present invention means products which are not completely finished, such as emulsions or preparations, which are further processed into finished products at a later stage. In this one or more final processing steps, further essential compounds may be added to the semi-finished product, dilution steps may take place or the semi-finished product may be subjected to various mechanical processes in order to achieve certain macroscopic properties. In this context, semi-finished products refer to foods that are intended to be consumed only in a further processed state, e.g. after the addition of aroma or flavour substances that are (partially) decisive for the sensory impression.

Another aspect of the present invention relates to a process for producing an emulsion according to the invention, comprising or consisting of the following steps:
Providing an oil phase containing an emulsifier precursor according to the invention or an emulsifier according to the invention or a mixture thereof, and an aqueous phase,
optionally: separate heating of the oil phase and the aqueous phase, preferably to a temperature in the range of 40 to 100° C., preferably in the range of 60 to 85° C.,
optionally: adding one or more different compounds to lower or increase the viscosity to the heated oil phase,
Mixing the optionally heated aqueous phase and the optionally heated oil phase, and
optionally: Cool while stirring.

“Providing” for the purpose of preparing the emulsions according to the present invention means placing the respective phases in a suitable container or a plurality of such containers, preferably under controlled conditions comprising pressure and temperature, preferably with the aim of enabling the subsequent process steps for preparing emulsions according to the invention.

In some oil phases or aqueous phases according to the invention, it is advantageous or necessary to heat the respective provided phases in order to ensure the production of an emulsion according to the invention. The heating step usually serves to homogenise or contribute to the homogenisation of the respective phase. This step can be additionally promoted or supported, for example, by stirring or other measures for promoting convection known to the skilled person from the prior art.

“Mixing” in the sense of the present invention describes the combining of compounds so that a mixture that is as uniform as possible, i.e. homogeneity, is achieved. For mixing, it is necessary that a relative movement of partial volume elements of the compounds to be mixed is present. This can be due to diffusion, convection or mechanical energy introduced from outside. Mixing serves, among other things, the production of solutions, emulsions and/or heat transport. During mixing, it is not uncommon for temperature changes to occur in the system, especially when mixing liquids, without chemical reactions taking place (due to the mixing enthalpy). In addition, volume contractions or increases can occur. Mixing is carried out, inter alia, by stirring, emulsifying, dissolving, ultrasonic action depending on the state of aggregation and the properties of the components to be mixed. In principle, mixing devices (mixers) can be divided into static and dynamic mixers. While the former act through turbulence that is created on specially shaped structures in liquids or gases as they flow through, the latter is actively generated in dynamic mixers. Mixer types include propeller, turbo, blade, trough, planetary, friction, screw, roller, centrifugal, counterflow, jet, drum, cone, tumbling, gyroscopic, cooling, vacuum, flow, gravity, fluid and pneumatic mixers.

“Cooling” in the sense of the present invention is the optional cooling of the mixture obtained from the preceding steps, if prepared at elevated temperatures compared to room temperature or standard conditions, to a lower temperature, preferably back to room temperature or the temperature at standard conditions. Preferably, cooling is carried out using coolers or heat exchangers known in the prior art. Likewise, mixing or stirring may promote cooling of the mixture as described in the preceding paragraph.

The present invention is explained in more detail below with reference to selected examples, although the present invention is not limited thereto.

Unless otherwise stated, all of the following (%) figures refer to weight and are to be understood as “ wt.-%”, based on the total weight of the respective composition or the respective emulsifier or other specified total reference.

### EXAMPLE 1

### Exemplary Fatty Acid Compositions (As Starting Material)

Table 1 shows an overview of oils used as examples and their composition with regard to their fatty acid residues (FS). The information of the type “Cx:y” in the left column (“FS”) stands for the number of carbon atoms x and the number of double bonds y in the respective fatty acid residue. For example, “C18:1” means a fatty acid residue with 18 carbon atoms and one double bond. The trivial name of the underlying acid is given in parentheses.

TABLE 12
Composition in terms of fatty acid
residues of the fatty acid compositions used.

| | | Palm | | | | Sun- |
|---|---|---|---|---|---|---|
| FS | Babassu | kernel | Coconut | Macaúba | Neutral | flower |
| C6:0 | 0.3% | 0.2% | 0.6% | 0.18% | 0.03% | —/— |
| (caproic acid) | | | | | | |
| C8:0 | 5.6% | 3.2% | 7.3% | 5.61% | 48.3% | 0.04% |
| (caprylic acid) | | | | | | |
| C10:0 | 5.3% | 3.2% | 5.9% | 4.9% | 33.3% | 0.03% |
| (capric acid) | | | | | | |
| C12:0 | 46.5% | 45.3% | 47.9% | 43.5% | 0.09% | 0.01% |
| (lauric acid) | | | | | | |
| C14:0 | 17.5% | 15.6% | 19.0% | 10.6% | —/— | 0.07% |
| (myristic acid) | | | | | | |
| C16:0 | 9.8% | 8.7% | 8.5% | 8.0% | —/— | 6.8% |
| (palmitic acid) | | | | | | |
| C18:0 | 3.9% | 2.4% | 3.2% | 3.8% | —/— | 3.7% |
| (stearic acid) | | | | | | |
| C18:1 | 14.9% | 15.3% | 4.7% | 19.7% | —/— | 31.3% |
| (Oleic acid) | | | | | | |
| C18:2 | 2.4% | 2.4% | 0.7% | 2.3% | —/— | 61.4% |
| (linoleic acid) | | | | | | |

### EXAMPLE 2

### Proportions of Mono-Glyceride Citrate (MGC) and Diglyceride Citrate (DGC) in Reference Samples and Emulsifiers According to the Invention

Citric acid esters of mono- and diglycerides of edible fatty acids have been known for a long time and are marketed under the name E472c, among others. Compounds consisting of a fatty acid or fatty acid residue, one or more glycerol units, which in turn may be linked via one or more ether bonds, and a citric acid or citric acid residue are referred to as mono-glyceride citrates (MGC). Compounds with two fatty acids or fatty acid residues, one or more glycerol units, which in turn may be linked via one or more ether bonds, and a citric acid or citric acid residue are referred to as diglyceride citrates (DGC). The herein described results of the HPLC-MS analysis of emulsifiers according to the invention and other reference samples (see Table 2) show that the proportion of MGC and DGC is responsible for the actual emulsifying effect in cosmetic formulations.

TABLE 34
Proportions of MGC and DGC in reference samples and
emulsifiers according to the invention.

| | | (Comparison) | | | |
|---|---|---|---|---|---|
| | | sample/ | MGC/ | DGC/ | Total/ |
| Designation | Raw material | name | [%] | [%] | [%] |
| Dracorin CE | Glyceryl stearate | Sample 1 | 7.6 | 2.16 | 10 |
| | citrate | | | | |
| Dracorin GOC | Glyceryl oleate | Sample 2 | 8.3 | 7.3 | 16 |
| | citrate | | | | |
| Eco_Emuls | Citric acid ester | Eco | 7.2 | 18.9 | 26 |
| Palmk. | mono-and | Emulsifier 1 | | | |
| | diglyceride, | | | | |
| | (Palm kernel oil | | | | |
| | based) | | | | |
| Eco_Emuls | Citric acid ester | Eco | 8.3 | 17.7 | 26 |
| Babassu | mono-and | Emulsifier 2 | | | |
| (laboratory | diglyceride | | | | |
| experiment) | (Babassu oil based) | | | | |
| Eco_Emuls | Citric acid ester | Eco | 9 | 19 | 28 |
| coco | mono-and | Emulsifier 3 | | | |
| (laboratory | diglyceride | | | | |
| experiment) | (coconut oil based) | | | | |

Comparatively analysed emulsifier samples not falling into the category of emulsifiers according to the invention showed percentages of MGC and DGC in ranges significantly different from those of emulsifiers according to the invention.

Typical emulsifiers known from the prior art based on glyceryl stearate citrates have, for example, according to own investigations, a content of MGC in the range of 7.6 to 20.4 wt.-%, a content of DGC in the range of 1.0 to 2.6 wt.-% and, with regard to the sum of the proportions of MGC and DGC, a content in the range of 10 to 15 wt.-%, based on the total weight of the emulsifier.

Typical emulsifiers known from the prior art based on citric acid ester mono- and diglycerides of palm and sunflower oil have, for example, according to own investigations, a content of MGC in the range of 5 to 7.2 wt.-%, a content of DGC in the range of 3.9 to 14.2 wt.-% and, with regard to the sum of the proportions of MGC and DGC, a content in the range of 11 to 19 wt.-%, based on the total weight of the emulsifier.

Typical emulsifiers known from the prior art based on glyceryl oleate citrates have, for example, according to own investigations, a content of MGC in the range of 8.3 to 11.2 wt.-%, a content of DGC in the range of 6.2 to 7.3 wt.-% and, with regard to the sum of the proportions of MGC and DGC, a content in the range of 16 to 17 wt.-%, based on the total weight of the emulsifier.

“Eco emulsifiers” in the tables shown herein denote exemplary emulsifiers according to the invention. The fatty acid composition of the final emulsifier or “eco emulsifier” according to the invention reflects the fatty acid distribution of the starting materials. Normalised to 100 wt.-%, i.e. with respect to the sum of the proportions of the weight of the respective fatty acids or fatty acid residues, the “eco emulsifiers” according to the invention are characterised by the following fatty acid composition (allocation of the fatty acids according to the explanations in example 1).

Eco Emulsifier 1: 0.19 wt.-% C6:0, 3.36 wt.-% C8:0, 3.35 wt.-% C10:0, 47.30 wt.-% C12:0, 16.25 wt.-% C14:0, 9.07 wt.-% C16:0, 2.52 wt.-% C18:0, 15.62 wt.-% C18:1 and 2.27 wt.-% C18:2.

Eco Emulsifier 2: 0.27 wt.-% C6:0, 5.10 wt.-% C8:0, 4.94 wt.-% C10:0, 43.8 wt.-% C12:0, 16.65 wt.-% C14:0, 9.29 wt.-% C16:0, 3.68 wt.-% C18:0, 14.14 wt.-% C18:1 and 2.17 wt.-% C18:2.

Eco Emulsifier 3: 0.35 wt.-% C6:0, 4.97 wt.-% C8:0, 3.95 wt.-% C10:0, 38.6 wt.-% C12:0, 21.36 wt.-% C14:0, 13.03 wt.-% C16:0, 2.58 wt.-% C18:0, 13.00 wt.-% C18:1 and 3.23 wt.-% C18:2.

The “Eco Emulsifiers” exemplified or examined herein can preferably be prepared by the processes described in Examples 5 and 6 below, substituting babassu oil for the respective specified “raw material”. For example, to obtain the “coconut oil-based Eco Emulsifier 3”, one of the processes according to the invention of Examples 5 or 6 is to be carried out using coconut oil instead of the babassu oil used therein.

### EXAMPLE 3

### Methods for Determining the Content of MGC and DGC

As described above, the MGC and DGC content of the emulsifiers of the invention and of the reference samples can preferably be determined, inter alia, by two methods.

In the first method, the coupling of liquid chromatography (HPLC) with mass spectrometry (MS) is used for evaluation, the so-called HPLC-MS. The assignment of the peaks in the MS obtained spectra is done via the mass and retention indices. The quantity is estimated by integrating the respective peaks (so-called area-%).

The second method used is based on precipitation and gravimetric analysis.

A comparison of the amounts of MGC and DGC from the two previously mentioned methods (see Table 3) shows that the values agree within usual error limits.

TABLE 5
Comparison of methods HPLC-MS vs. gravimetry for the
determination of the proportions of MGC and DGC in
(further) emulsifiers according to the invention.

| | | | | Citrate | Gravi- |
|---|---|---|---|---|---|
| | | MGC/ | DGC/ | total/ | metric/ |
| Raw material | Sample/Name | [%] | [%] | [%] | [%] |
| Citric acid ester | Eco Emulsifier 4 | 8.25 | 17.2 | 25.45 | 23 |
| mono-and | | | | | |
| diglyceride | | | | | |
| (Babassu oil based) | | | | | |
| Citric acid ester | Eco Emulsifier 5 | 7.18 | 18.9 | 26.08 | 24 |
| mono-and | | | | | |
| diglyceride | | | | | |
| (Palm kernel oil | | | | | |
| based) | | | | | |
| Citric acid ester | Eco Emulsifier 6 | 8.4 | 21.5 | 29.9 | 30 |
| mono-and | | | | | |
| diglyceride | | | | | |
| (Coconut oil based, | | | | | |
| BRA) | | | | | |
| Citric acid ester | Eco Emulsifier 7 | 8.6 | 20.6 | 29.2 | 26 |
| mono-and | | | | | |
| diglyceride | | | | | |
| (coconut oil based, | | | | | |
| PHL) | | | | | |
| Mono-and | Eco Emulsifier 8 | 12.8 | 19.8 | 32.6 | 27 |
| diglyceride | | | | | |
| (Coconut oil based, | | | | | |
| LKA) | | | | | |

In some cases, the country of origin of the underlying oils is mentioned in the preceding table, wherein “BRA” stands for Brazil, “PHL” for the Philippines and “LKA” for Sri Lanka. The fatty acid composition of the final emulsifier or “eco emulsifier” reflects the fatty acid distribution of the starting materials. Normalised to the 100 wt.-%, i.e. with respect to the sum of the proportions of the weight of the respective fatty acids or fatty acid residues, the “Eco Emulsifiers” according to the invention are characterised by the following fatty acid composition (allocation of the fatty acids according to the explanations in Example 1).

Eco Emulsifier 4: 0.31 wt.-% C6:0, 5.09 wt.-% C8:0, 4.93 wt.-% C10:0, 44.23 wt.-% C12:0, 17.22 wt.-% C14:0, 9.92 wt.-% C16:0, 3.96 wt.-% C18:0, 12.87 wt.-% C18:1, 1.23 wt.-% C18:2.

Eco Emulsifier 5: 0.19 wt.-% C6:0, 3.30 wt.-% C8:0, 3.36 wt.-% C10:0, 48.71 wt.-% C12:0, 17.18 wt.-% C14:0, 9.83 wt.-% C16:0, 2.78 wt.-% C18:0, 15.01 wt.-% C18:1 and 1.96 wt.-% C18:2.

Eco Emulsifier 6: 0.38 wt.-% C6:0, 5.21 wt.-% C8:0, 4.16 wt.-% C10:0, 40.78 wt.-% C12:0, 21.72 wt.-% C14:0, 12.56 wt.-% C16:0, 2.21 wt.-% C18:0, 10.4 wt.-% C18:1 and 2.45 wt.-% C18:2.

Eco Emulsifier 7: 0.48 wt.-% C6:0, 7.16 wt.-% C8:0, 5.99 wt.-% C10:0, 49.20 wt.-% C12:0, 19.60 wt.-% C14:0, 8.89 wt.-% C16:0, 3.37 wt.-% C18:0, 4.64 wt.-% C18:1 and 0.68 wt.-% C18:2.

Eco Emulsifier 8: 0.51 wt.-% C6:0, 7.69 wt.-% C8:0, 5.79 wt.-% C10:0, 48.90 wt.-% C12:0, 20.20 wt.-% C14:0, 8.06 wt.-% C16:0, 3.32 wt.-% C18:0, 4.64 wt.-% C18:1 and 0.68 wt.-% C18:2.

The “Eco Emulsifiers” exemplified or examined herein can preferably be prepared by the processes described in Examples 5 and 6 below, using the respective specified “raw material” instead of babassu oil. For example, to obtain the “coconut oil based eco emulsifier 6”, one of the processes according to the invention of examples 5 or 6 is to be carried out using coconut oil instead of babassu oil used therein.

### EXAMPLE 4

### Relative Proportion of DGC to MGC in Emulsifiers According to the Invention

Emulsifiers according to the invention are preferably characterised by a certain ratio of DGC to MGC of the emulsifiers according to the invention, wherein the sum of the proportions of MGC and DGC have been normalised to 100% (cf. Table 4).

TABLE 67
Ratio DGC/MGC of exemplary emulsifiers according to the invention,
wherein the sum of the proportions of MGC and DGC
was normalised to 100%.

| | | | | Ratio |
|---|---|---|---|---|
| | | MGC/[%] | DGC/[%] | DGC/ |
| Raw material | Sample/Name | standardised | standardised | MGC |
| Citric acid ester | Eco Emulsifier 4 | 29.5 | 70.5 | 2.39 |
| mono-and | | | | |
| diglyceride | | | | |
| (Babassu oil | | | | |
| based) | | | | |
| Citric acid ester | Eco Emulsifier 5 | 30.1 | 69.9 | 2.32 |
| mono-and | | | | |
| diglyceride | | | | |
| (Palm | | | | |
| based) kernel oil | | | | |
| based) | | | | |
| Citric acid ester | Eco Emulsifier 6 | 28.5 | 71.5 | 2.51 |
| mono-and | | | | |
| diglyceride | | | | |
| (Coconut oil | | | | |
| based, BRA) | | | | |
| Citric acid ester | Eco Emulsifier 7 | 32.3 | 67.7 | 2.10 |
| mono-and | | | | |
| diglyceride | | | | |
| (coconut oil | | | | |
| based, PHL) | | | | |
| Citric acid ester | Eco Emulsifier 8 | 28.9 | 71.1 | 2.46 |
| mono-and | | | | |
| diglyceride | | | | |
| (Coconut oil | | | | |
| based, LKA) | | | | |

The ratio of the proportions of the total amount of compounds of formula (I) and one or more salt(s) of compound(s) of formula (I) with exactly one fatty acid residue attached to the glycerol backbone by an ester bond, i.e. MGC, to the total amount of compounds of formula (I) and one or more salt(s) of compound(s) of formula (I) having exactly two fatty acid residues attached to the glycerol backbone by ester bonds, i.e. DGC, relative to the weight of these compounds, is in the range from 1:1.5 to 1:3, preferably in the range from 1:1.75 to 1:2, particularly preferably in the range from 1:2 to 1:2.5.

### EXAMPLE 5

### Presentation of a Liquid Emulsifier According to the Invention: Variant 1

In a heated double jacket vessel with a leaf stirrer, 900 g (˜1.35 mol) of Babassu oil was mixed with 189 g (2.05 mol) of glycerol as described herein and heated to 50° C. An aqueous solution of sodium hydroxide (10 mol-%) was added and the reaction mixture was heated to 250° C. and stirred at this temperature for 120 min. Any process water formed was removed by applying a vacuum of 700 mbar. The obtained reaction mixture was cooled, toluene was added and the mixture was extracted with water (twice). The organic phase was separated and after removal of the solvent yielded the emulsifier precursor (1019 g, ˜3.6 mol, 89%) with adequate ratios of mono-, di- and triglycerides.

In a heated double jacket vessel with leaf stirrer, 1019 g (˜3.60 mol) emulsifier precursor was mixed with citric acid (691 g, 3.6 mol) and heated to 155° C. for 4 h while stirring. Any process water formed was removed from the reaction mixture by applying a vacuum of 800 mbar. After cooling to room temperature, toluene and water were added to the reaction mixture, the phases were separated and the organic phase was washed with water (twice) and neutralised by adding a solution of alkali hydroxide (10 mol-%, NaOH/KOH). The organic phase was separated and, after removal of the solvent according to the HPLC-MS analysis described herein, yielded 990 g of an emulsifier according to the invention containing more than 26 wt.-% of citric acid esters and ethers according to the compounds of formula (I), based on the total weight of the emulsifier. The emulsifier obtained corresponds to “Eco Emulsifier 4”.

### EXAMPLE 6

### Presentation of a Solid Emulsifier According to the Invention: Variant 2

In a heated double jacket vessel with a leaf stirrer, 900 g (˜1.35 mol) of Babassu oil was mixed with 189 g (2.05 mol) of glycerol as described herein and heated to 50° C. An aqueous solution of sodium hydroxide (10 mol-%) was added and the reaction mixture was heated to 250° C. and stirred at this temperature for 120 min. Any process water formed was removed by applying a vacuum of 700 mbar. The reaction mixture obtained was cooled, toluene was added and the mixture was extracted with water (twice). The organic phase was separated and after removal of the solvent yielded the emulsifier precursor according to the invention (1019 g, ˜3.6 mol, 89%) with adequate ratios of mono-, di- and triglycerides.

In a heated double jacket vessel with leaf stirrer, 1019 g (˜3.60 mol) emulsifier precursor was mixed with citric acid (691 g, 3.6 mol) and heated to 155° C. for 4 h while stirring. Any process water formed was removed from the reaction mixture by applying a vacuum of 800 mbar. After cooling to room temperature, toluene and water were added to the reaction mixture, the phases were separated and the organic phase was washed with water (twice) and neutralised by adding a solution of alkali hydroxide (10 mol-%, NaOH/KOH). The phases were separated and the organic phase was freed from the solvent. The obtained residue was taken up in 1000 g alcohol (methanol, ethanol, propanol) and stirred for 2 h. After cooling and filtration, 260 g of a solid emulsifier containing more than 25 wt.-% of citric acid esters and ethers was obtained, based on the total weight of the emulsifier. The emulsifier obtained by this method corresponds to “Eco Emulsifier 4” (enriched). Optionally, by repeated washing with methanol/water, the proportion of citric acid esters and ethers according to compounds of formula (I) could be increased to 90-100 wt.-%, based on the total weight of the emulsifier. By using different natural oils as described herein and in the above-mentioned formulation, it was possible to produce “Eco Emulsifier 5”, “Eco Emulsifier 6” and “Eco Emulsifier 7”. Instead of the process parameters mentioned above (pressure, temperature, solvent, (substance) quantity ratios, etc.), further combinations of process parameters mentioned herein also result in emulsifier precursors or emulsifiers according to the invention, wherein the individual process parameters can be selected independently of one another.

### APPLICATION EXAMPLE 1

### Proof of Emulsifying Properties

A direct indication of the effectiveness of the emulsifying effect of the emulsifiers according to the invention is provided, for example, by the volume-based particle size determination of the emulsions by means of laser diffraction (measuring device: e.g. Malvern Mastersizer 3000). Smaller oil droplets in the emulsions according to the invention indicate a higher reduction of the surface tension by the emulsifier. Emulsions with small particle sizes are physically more stable, show improved sensorial properties in terms of better spreadability on the skin as well as a whiter and shinier appearance.

For these tests (see Table 5) an emulsifier precursor (tests No. 1 and 5), the emulsifier with 85-100 wt.-% active content, i.e. with 85-100 wt.-% of the compounds of formula (I) based on the total weight of the emulsifier, (tests No. 2 and 6), combinations thereof (tests No. 3, 7, 9 and 10) and the emulsifier (tests No. 4 and 8) were used. The starting products for these substances were babassu oil and coconut oil, respectively. In test no. 1, emulsifiers according to the invention or emulsifiers known from the prior art were not used. Details on the composition of the emulsifiers, precursors, etc. used are described below (see Table 6).

TABLE 8
Test setup o/w emulsions.

| | Emulsifier/precursor based on | | | Emulsifier/precursor based on | | |
|---|---|---|---|---|---|---|
| | coconut oil/(w/w %) | | | babassu oil/(w/w %) | | |
| | Emulsifier | Emulsifier | Emul- | Emulsifier | Emulsifier | Emul- |
| No. | precursor | enriched | sifier | precursor | enriched | sifier |
| 1 | 0.75 | — | — | — | — | — |
| 2 | — | 0.25 | — | — | — | — |
| 3 | 0.75 | 0.25 | — | — | — | — |
| 4 | — | — | 1.0 | — | — | — |
| 5 | — | — | — | 0.75 | — | — |
| 6 | — | — | — | — | 0.25 | — |
| 7 | — | — | — | 0.75 | 0.25 | — |
| 8 | — | — | — | — | — | 1.0 |
| 9 | 0.75 | — | — | — | 0.25 | — |
| 10 | — | 0.25 | — | 0.75 | — | — |
| 11 | — | — | — | — | — | — |

Emulsions No. 1 and 5 from Table 5 and Table 6, respectively, showed the largest oil droplets, i.e. the emulsifier precursors used in these formulations showed a lower emulsifying effect compared to the other emulsifiers according to the invention. Emulsions No. 2, 3, and 4 (mixtures of enriched emulsifier and the emulsifier precursor or the coconut oil-based emulsifier) showed oil droplets of significantly smaller size, indicating a very good emulsifying effect. The results for emulsions no. 6, 7 and 8 from (based on babassu oil) are comparably good. In test no. 9, the enriched emulsifier from babassu oil is combined with the emulsifier precursor from coconut oil. In test no. 10, it is the other way round. Also in these two tests, the very good emulsifying effect was shown by small oil droplets. It can be concluded that the citrate esters are responsible for the particularly beneficial emulsifying effect.

The following formulations in Table 6 were used for the tests or O/W emulsions 1 to 11 from application example 1.

TABLE 6
Recipes for tests 1 to 11 from application example 1.

| | Proportion in emulsion or test/[%] | | | | | | | | | | |
|---|---|---|---|---|---|---|---|---|---|---|---|
| Substance | 1 | 2 | 3 | 4 | 5 | 6 | 7 | 8 | 9 | 10 | 11 |
| Phase A | | | | | | | | | | | |
| Emulsifier | 0.75 | — | 0.75 | — | — | — | — | — | 0.75 | — | — |
| precursor | | | | | | | | | | | |
| (coconut oil) | | | | | | | | | | | |
| Emulsifier, | — | 0.25 | 0.25 | — | — | — | — | — | — | 0.25 | — |
| enriched | | | | | | | | | | | |
| (coconut oil) | | | | | | | | | | | |
| Final | — | — | — | 1.0 | — | — | — | — | — | — | — |
| emulsifier | | | | | | | | | | | |
| (coconut oil) | | | | | | | | | | | |
| Emulsifier | — | — | — | — | 0.75 | — | 0.75 | — | — | 0.75 | — |
| precursor | | | | | | | | | | | |
| (Babassu oil) | | | | | | | | | | | |
| Emulsifier, | — | — | — | — | | 0.25 | 0.25 | — | — | — | — |
| enriched | | | | | | | | | | | |
| (babassu oil) | | | | | | | | | | | |
| Final | — | — | — | — | — | — | — | 1.0 | 0.25 | — | — |
| emulsifier | | | | | | | | | | | |
| (babassu oil) | | | | | | | | | | | |
| Cetylstearyl | 1.0 | 1.0 | 1.0 | 1.0 | 1.0 | 1.0 | 1.0 | 1.0 | 1.0 | 1.0 | 1.0 |
| alcohol | | | | | | | | | | | |
| Pentaerythrityl | 1.2 | 1.2 | 1.2 | 1.2 | 1.2 | 1.2 | 1.2 | 1.2 | 1.2 | 1.2 | 1.2 |
| distearate | | | | | | | | | | | |
| Cetearyl | 4.0 | 4.0 | 4.0 | 4.0 | 4.0 | 4.0 | 4.0 | 4.0 | 4.0 | 4.0 | 4.0 |
| octanoate | | | | | | | | | | | |
| Persea | 2.0 | 2.0 | 2.0 | 2.0 | 2.0 | 2.0 | 2.0 | 2.0 | 2.0 | 2.0 | 2.0 |
| Gratissinna | | | | | | | | | | | |
| (Avocado) Oil | | | | | | | | | | | |
| Capryl/Caprin/ | 6.0 | 6.0 | 6.0 | 6.0 | 6.0 | 6.0 | 6.0 | 6.0 | 6.0 | 6.0 | 6.0 |
| Triglceride | | | | | | | | | | | |
| Dimethicone | 0.3 | 0.3 | 0.3 | 0.3 | 0.3 | 0.3 | 0.3 | 0.3 | 0.3 | 0.3 | 0.3 |
| Phase B | | | | | | | | | | | |
| Carbomer | 0.2 | 0.2 | 0.2 | 0.2 | 0.2 | 0.2 | 0.2 | 0.2 | 0.2 | 0.2 | 0.2 |
| (Ultrez-10) | | | | | | | | | | | |
| Xanthan gum | 0.1 | 0.1 | 0.1 | 0.1 | 0.1 | 0.1 | 0.1 | 0.1 | 0.1 | 0.1 | 0.1 |
| Phase C | | | | | | | | | | | |
| Water, | Supplement up to 100% | | | | | | | | | | |
| demineralised | | | | | | | | | | | |
| Hydroxy- | 0.7 | 0.7 | 0.7 | 0.7 | 0.7 | 0.7 | 0.7 | 0.7 | 0.7 | 0.7 | 0.7 |
| acetophenone | | | | | | | | | | | |
| Glycerine 99% | 3.0 | 3.0 | 3.0 | 3.0 | 3.0 | 3.0 | 3.0 | 3.0 | 3.0 | 3.0 | 3.0 |
| Phase D | | | | | | | | | | | |
| Sodium | 0.4 | 0.4 | 0.4 | 0.4 | 0.4 | 0.4 | 0.5 | 0.4 | 0.4 | 0.4 | 0.3 |
| hydroxide, | | | | | | | | | | | |
| 10% aqueous | | | | | | | | | | | |
| solution | | | | | | | | | | | |

| | |
|---|---|
| Total | 100.0 |

Note on production:
Heat phase A and C separately to approx. 80° C.
Remove phase A from the heat source, add phase B and disperse using a magnetic stirrer.
Add phase C to phase AB and emulsify. (Ultra Turray stirrer, 3 minutes 6000 rpm).
Add phase D while stirring and allow cooling using a leaf stirrer.
Adjust the pH value to 5.8.

Emulsifier precursors or emulsifiers according to the invention (printed in bold in “Phase A” of Table 6) can preferably be obtained according to one of the processes of Examples 5 or 6 or their corresponding analogues with regard to the use of the respective starting material (coconut oil or babassu oil). In addition, as described herein, combinations according to the invention other than those of the process parameters explicitly described in Examples 5 and 6 (pressure, temperature, solvent, (substance) quantity ratios, etc.) can also lead to emulsifier precursors or emulsifiers according to the invention.

The results of the volume-related particle size measurement are given as Dv_{0.5} or Dv_{0.9} values in μm. For example, a Dv_{0.5} of 11.7 μm indicates for example that 50% of the particles are smaller than 11.7 μm, or a Dv_{0.9} of 23.3 μm indicates that 90% of the particles are smaller than 23.3 μm.

The results of the particle size measurement of emulsions 1 to 11 from Table 6 carried out by means of laser diffraction (Malvern Mastersizer 3000) are summarised in Table 7. Procedures for performing laser diffraction tests are well known to the skilled person. Preferably, the samples investigated were filtered through a suitable syringe filter to avoid contamination of the sample (e.g. dust).

TABLE 7
Results of the particle size measurement of emulsions 1 to 11 from Table 6
carried out by means of laser diffraction.

| No. | D _{v0.5}/[μm] | D _{v0.9}/[μm] |
|---|---|---|
| 1 | 11.7 | 23.3 |
| 2 | 5.4 | 9.4 |
| 3 | 4.6 | 7.8 |
| 4 | 3.3 | 5.7 |
| 5 | 13.2 | 30.8 |
| 6 | 6.1 | 10.6 |
| 7 | 5.1 | 9.1 |
| 8 | 4.1 | 6.6 |
| 9 | 4.7 | 8.6 |
| 10 | 4.6 | 7.9 |
| 11 | 12.3 | 29.1 |

### APPLICATION EXAMPLE 2

### Emulsions with Citrate Esters with High Proportions of C12/C14 Fatty Acid Esters (Citrate Ester Babassu Oil- or Coconut Oil-Based) Compared to Emulsifiers Based on C16/C18 Fatty Acids (Glyceryl Stearate Citrate/glyceryl Oleate Citrate)

Emulsions according to the invention, comprising emulsifiers according to the invention, i.e. citrate esters with high proportions of C12 and C14 fatty acid residues based on babassu oil or, respectively, coconut oil are compared to emulsifiers based on C16 and C18 fatty acids (glyceryl stearate citrate/glyceryl oleate citrate).

The following formulations in Table 8 were used for the tests or O/W emulsions A to E from application example 2.

TABLE 8
Recipe for the tests or O/W emulsions from application example 2.

| | Proportion in emulsion or test/[%] | | | | |
|---|---|---|---|---|---|
| Substance | A | B | C | D | E |
| Phase A | | | | | |
| Glyceryl stearate citrate | 1.0 | — | | — | — |
| Glyceryl oleate citrate | — | 1.0 | | — | — |
| Citrate ester (babassu oil based) | — | — | 1.0 | | — |
| Citrate ester (babassu oil based) mixed | — | — | — | 1.0 | — |
| with sunflower oil 1:1 (w/w %) | | | | | |
| Citrate ester (coconut oil based) | — | — | — | — | 1.0 |
| Cetylstearyl alcohol | 1.0 | 1.0 | 1.0 | 1.0 | 1.0 |
| Pentaerythrityl distearate | 1.2 | 1.2 | 1.2 | 1.2 | 1.2 |
| Cetearyl octanoate | 4.0 | 4.0 | 4.0 | 4.0 | 4.0 |
| Persea Gratissima (Avocado) Oil | 2.0 | 2.0 | 2.0 | 2.0 | 2.0 |
| Capryl/Caprin/Triglceride | 6.0 | 6.0 | 6.0 | 6.0 | 6.0 |
| Dimethicone | 0.3 | 0.3 | 0.3 | 0.3 | 0.3 |
| Phase B | | | | | |
| Carbomer (Ultrez-10) | 0.2 | 0.2 | 0.2 | 0.2 | 0.2 |
| Xanthan gum | 0.1 | 0.1 | 0.1 | 0.1 | 0.1 |
| Phase C | | | | | |
| Water, demineralised | Supplement up to 100% | | | | |
| Hydroxyacetophenone | 0.7 | 0.7 | 0.7 | 0.7 | 0.7 |
| Glycerine 99% | 3.0 | 3.0 | 3.0 | 3.0 | 3.0 |
| Phase D | | | | | |
| Sodium hydroxides, 10% aqueous | 0.4 | 0.4 | 0.4 | 0.4 | 0.4 |
| solution. | | | | | |

| | |
|---|---|
| Total | 100.0 |

Note on Production:
Heat phase A and C separately to approx. 80° C.
Remove phase A from the heat source, add phase B and disperse using a magnetic stirrer.
Add phase C to phase AB and emulsify. (Ultra Turrax stirrer, 3 minutes 6000 rpm).
Add phase D while stirring and allow cooling using a leaf stirrer.
Adjust the pH value to 5.8.

The results of the particle size measurements of emulsions A to E of Table 8 carried out by laser diffraction (Malvern Mastersizer 3000) are summarised in Table 9.

TABLE 9
Results of the particle size measurement of emulsions A to E of table 8,
carried out by means of laser diffraction.

| No. | Emulsifier | Conc. (%) | D _{v0.5} (μm) | D _{v0.9} (μm) |
|---|---|---|---|---|
| A | Glyceryl stearate citrate | 1.0 | 5.9 | 10.7 |
| B | Glyceryl oleate citrate | 1.0 | 5.4 | 9.4 |
| C | Citrate ester (babassu oil | 1.0 | 3.9 | 5.9 |
| | based) | | | |
| D | Citrate ester (Babassu oil | 1.0 | 4.9 | 8.3 |
| | based) mixed with sunflower | | | |
| | oil; 1:1 (w/w %) | | | |
| E | Citrate ester (coconut oil) | 1.0 | 3.4 | 5.4 |

The emulsions C, D and E according to the invention containing one or more emulsifiers according to the invention show smaller oil droplets in comparison to emulsions A and B. Accordingly, the emulsifiers according to the invention show an improved emulsifying effect compared to conventional emulsifiers.

1. An emulsifier comprising:
a mixture of compounds of formula (I), and/or salts thereof: 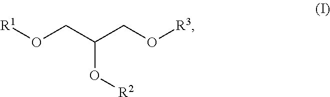
wherein in each case applies:
R¹, R², and R³ are independently chosen from:
(i) a hydrogen atom,
(ii) a fatty acid residue attached to the glycerol backbone by an ester bond,
(iii) a citric acid residue attached to the glycerol backbone by an ester bond or an ether bond,
with the proviso that in each case at most one of R¹, R², and R³ is (iii) a citric acid residue attached to the glycerol backbone by an ester bond or ether bond,
wherein the total amount of the compounds of formula (I) and salts thereof having one or two fatty acid residues (ii) attached to the glycerol backbone by an ester bond is higher than 40 wt.-%, based on the total weight of the emulsifier,
the total amount of the compounds of formula (I) and salts thereof having exactly one citric acid residue (iii) attached to the glycerol backbone by an ester bond or ether bond and at least one fatty acid residue (ii) attached to the glycerol backbone by an ester bond is higher than 20 wt.-%, based on the total weight of the emulsifier, and
the total amount of the compounds of formula (I) and salts thereof having a fatty residue (ii) attached to the glycerol backbone, wherein the fatty acid is chosen from caproic acid, caprylic acid, capric acid, lauric acid, and myristic acid, is higher than 55 wt.-%, based on the total weight of the compounds of formula (I), and/or the total amount of the compounds of formula (I) and salts thereof having a fatty residue (ii) attached to the glycerol backbone, wherein the fatty acid is chosen from lauric acid and myristic acid, is higher than 50 wt.-%, based on the total weight of the compounds of formula (I), and/or the total amount of the compounds of formula (I) having a fatty acid residue (ii) attached to the glycerol backbone, wherein the fatty acid is chosen from palmitic acid, palmitoleic acid, stearic acid, oleic acid, linoleic acid, and linolenic acid, is lower than 40 wt. % based on the total weight of the compounds of formula (I).

2. The emulsifier according to claim 1, wherein the mixture of the compounds of formula (I) comprises 5-20% of compounds of formula (I) having exactly one fatty acid residue and at most one citric acid residue, and 20-50% of compounds of formula (I) having exactly two fatty acid residues and at most one citric acid residue, and 25-60% of compounds of formula (I) having exactly three fatty acid residues.

3. A method for preparing an emulsifier according to claim 1, comprising:
(A) reacting a fatty acid composition comprising one or more fatty acids and/or one or more fatty acid residues, with glycerol and one or more catalytic base(s),
(B) adding citric acid and/or a salt thereof to the mixture obtained in step (A),
(C) optionally, precipitating the mixture obtained in step (B) with one or more solvents to enrich the compound(s) of formula (I), and
(D) optionally, purifying the mixture obtained in step (B) or (C) using one or more solvents selected from the group consisting of halogenated, polar, protic polar, protic non-polar, “green” solvents and mixtures thereof and/or performing one or more purification process(es) selected from the group consisting of filtration, ultra- or nanofiltration, adsorption and absorption techniques, extraction, recrystallisation and chromatographic methods and combinations thereof,
wherein in the fatty acid composition used in step (A), the total amount of fatty acids selected from the group consisting of caproic acid, caprylic acid, capric acid, lauric acid and myristic acid and/or fatty acid residues selected from the group consisting of the fatty acid residue of caproic acid, the fatty acid residue of caprylic acid, the fatty acid residue of capric acid, the fatty acid residue of lauric acid and the fatty acid residue of myristic acid is higher than 45 wt.-%,

4. The method according to claim 3, wherein in step (A) glycerol is used for the reaction in an amount of 0.5 to 5 mol equivalents, based on the total amount of substance of fatty acid composition, and/or
the one or more catalytic base(s) from step (A) is/are used in an amount of 0.01 to 0.5 mol equivalents, based on the total amount of substance of the fatty acid composition, and/or
the one or more catalytic base(s) of step (A) is/are independently selected from the group consisting of amine bases, amino acids, Brønsted bases, Lewis bases, alkali salts, alkaline earth salts, and mixtures thereof.

5. The method according to claim 3, wherein step (A) is carried out at a temperature in the range of from 150 to 300° C., and/or wherein in step (B) a total amount of citric acid and/or salts thereof is in the range of from 0.1 to 2.0 parts by weight based on the total weight of the mixture obtained in step (A).

6. The method according to claim 1, wherein for the reaction taking place in step (B) after addition of citric acid and/or salts thereof, a temperature in the range of from 50 to 200° C. and optionally vacuum conditions is/are adjusted.

7. The method of claim 3, wherein the one or more solvent(s) in step (C), if present, is/are selected from the group consisting of pentane, hexane, heptane, cyclopentane, cyclohexane, tetrachloromethane, diisopropyl ether, toluene, benzene, diethyl ether, dichloromethane, chloroform, acetone, dioxane, tetrahydrofuran, 2-methylthetrahydrofuran, tert-butyl methyl ether, ethyl acetate, dimethyl sulphoxide, acetonitrile, benzonitrile, pyridine, 2-propanol, ethanol, methanol, acetic acid, formic acid, water, and mixtures thereof, and/or
wherein the fatty acid composition is selected from the group consisting of babassu oil, palm kernel oil, coconut oil, macaúba oil, microalgae oil, neutral oil, and mixtures thereof.

8. An emulsifier precursor obtained by or obtainable by purifying the mixture obtained from step (B) according to claim 3 by using one or more solvents selected from the group consisting of halogenated, polar, protic polar, protic non-polar, “green” solvents and mixtures thereof, and/or by performing one or more purification process(es) selected from the group consisting of filtration, ultra- or nanofiltration, adsorption and absorption techniques, extraction, recrystallisation and chromatographic methods and combinations thereof.

9. The emulsifier obtained by or obtainable by a process according to claim 3 wherein the process comprises step (C), and optionally wherein the proportion of the total amount of compounds of formula (I) and/or salts thereof having exactly one citric acid residue (iii) attached to the glycerol backbone by an ester bond or ether bond is higher than 50 wt. %.

10. (canceled)

11. An emulsion comprising
an oil phase comprising an emulsifier of claim 1 and
an aqueous phase.

12. (canceled)

13. A cleansing, cosmetic, or pharmaceutical preparation, or a preparation for consumption, comprising an emulsifier of claim 1.

14. A method of producing an emulsion according to claim 11 comprising:
providing an oil phase comprising the emulsifier of claim 1 and an aqueous phase,
optionally, separate heating of the oil phase and the aqueous phase,
optionally, adding one or more different compounds to lower or increase the viscosity to the heated oil phase,
mixing the optionally heated aqueous phase and the heated oil phase, and
optionally, cooling while stirring.

15. The emulsion of claim 1, wherein the total amount of the compounds of formula (I) and salts thereof having one or two fatty acid residues (ii) attached to the glycerol backbone by an ester bond is higher than higher than 90 wt.-%, based on the total weight of the emulsifier.

16. The emulsion of claim 15, wherein the total amount of the compounds of formula (I) and salts thereof having exactly one citric acid residue (iii) attached to the glycerol backbone by an ester bond or ether bond and at least one fatty acid residue (ii) attached to the glycerol backbone by an ester bond is higher than 20 wt.-%, based on the total weight of the emulsifier.

17. The emulsion of claim 1, wherein the total amount of the compounds of formula (I) and salts thereof having a fatty residue (ii) attached to the glycerol backbone, wherein the fatty acid is chosen from lauric acid and myristic acid, is higher than 60 wt.-%, based on the total weight of the compounds of formula (I).

18. The emulsion of claim 1, wherein the total amount of the compounds of formula (I) and salts thereof having a fatty residue (ii) attached to the glycerol backbone, wherein the fatty acid is chosen from palmitic acid, palmitoleic acid, stearic acid, oleic acid, linoleic acid, and linolenic acid, is lower than 30 wt.-%, based on the total weight of the compounds of formula (I).

19. The emulsifier according to claim 16, wherein the mixture of the compounds of formula (I) comprises 5-20% of compounds of formula (I) having exactly one fatty acid residue and at most one citric acid residue, and 20-50% of compounds of formula (I) having exactly two fatty acid residues and at most one citric acid residue, and 25-60% of compounds of formula (I) having exactly three fatty acid residues.
